(19) United States
(12) Patent Application Publication
(10) Pub. No.: US 2024/0079141 A1
(43) Pub. Date: Mar. 7, 2024

(54) SYSTEMS AND METHODS FOR STROKE DETECTION AT A FOOT SITE

(71) Applicant: KANDU HEALTH, INC., Campbell, CA (US)

(72) Inventors: Kirsten Carroll, San Francisco, CA (US); Lindsay Howard, San Jose, CA (US); Gerard von Hoffmann, Rancho Santa Fe, CA (US)

(21) Appl. No.: 18/241,761

(22) Filed: Sep. 1, 2023

Related U.S. Application Data
(60) Provisional application No. 63/374,574, filed on Sep. 5, 2022.

(51) Int. Cl. G16H 50/20; A43B 3/44

(52) U.S. Cl. CPC G16H 50/20; A43B 3/44

(57) ABSTRACT

A wearable device is described that is configured to be positioned on or adjacent to a lower extremity of a person and to monitor a plurality of skin surface sites of the person. The wearable device may include a stimulus source having a surface area to provide stimulus, a blood volume sensor configured to sense a stroke event at the at least one of the plurality of skin surface sites, an electrode configured to obtain bioelectrical data, and at least one processor communicatively coupled to the wearable device and configured to: output a first activation signal to the stimulus source, output a second activation signal to the blood volume sensor, receive an output signal from the blood volume sensor, and determine a stroke event at the at least one of the plurality of skin surface sites based on the output signal.

### CROSS-REFERENCE TO RELATED APPLICATIONS

This application is related to International Patent Application PCT/US2020/055604, filed Oct. 14, 2020, PCT/US2022/071701, filed Apr. 13, 2022, and claims the benefit of U.S. Provisional Application No. 63/374,574, filed Sep. 5, 2022, entitled “SYSTEMS AND METHODS FOR VASODILATION MONITORING AND CHARACTERIZATION,” the contents of which are herein incorporated by reference in their entirety.

### TECHNICAL FIELD

This disclosure relates generally to the field of wearable devices for physiological signal monitoring and, more specifically, to systems and methods for stroke detection at a foot site.

### BACKGROUND

Blood volume changes have pleiotropic effects in the body, and various physiological and environmental factors affect blood volume in a variety of tissues. Vasodilation refers to the dilation of blood capillaries near the skin. Vasodilation widens blood capillaries near the skin while constricting the deeper blood vessels. Further, vasodilation reduces the vascular resistance to blood flow, increases blood flow through the vessels near the skin, and decreases blood pressure inside the blood vessels near the skin. Vasodilation can be caused by temperature increases in the environment, parasympathetic nerve impulses, and changes in noradrenaline, histamine, prostaglandin, niacin, and carbon dioxide levels.

On the other hand, vasoconstriction refers to the constriction of blood capillaries near the skin. Vasoconstriction narrows the blood capillaries near the skin while dilating the deeper blood vessels. Additionally, vasoconstriction increases the vascular resistance to blood flow, decreases blood flow to near the skin, and increases the blood pressure inside the blood vessels near the skin. Vasoconstriction can be caused by temperature decreases in the environment, sympathetic nerve impulses, and changes in epinephrine, insulin, antidiuretic hormones, and oxygen levels.

Changes in vasoconstriction and/or vasodilation may be further indicative of impairments in the parasympathetic nervous system or sympathetic nervous system or other physiological changes that may be beneficial or harmful. As such, what is needed are improved systems for characterizing and/or monitoring changes in blood volumes at various tissues sites and understanding these blood volume changes in the larger context of the body's normal physiology and disease states.

### SUMMARY

Described herein are systems, devices, and methods for monitoring and/or characterizing vasodilation responses. In some instances, the systems, devices, and methods described herein may be used for multivariate detection of anomalous biologic events, biologic signals, and/or related data. In some implementations, the systems, devices, and methods are configured to detect a deviation from a predefined baseline. Multivariate may include using more than one, at least two, or a plurality of factors, markers, or other parameters to monitor responses, characterize responses, or detect anomalous biologic events or biologic signals. In some implementations, multivariate may include using one parameter measured at multiple locations or positions or at multiple times (e.g., random or fixed intervals, on demand, automatically, continuously, etc.). In various implementations, multivariate may include detecting a measured parameter, unilaterally or bilaterally, symmetrically or asymmetrically. The measured parameter may include a functional parameter (e.g., gait, speech, facial changes, etc.); a biological parameter or marker (e.g., blood proteins, metabolites, etc.); a quantitative parameter (e.g., limb asymmetry, heart rate variability, etc.); a spatial (e.g., neck vs. chest; arm vs. leg; etc.) difference in one or multiple (e.g., 2, 3, 4, 5, 10, 15, 20, etc.) measured parameters; and/or a temporal difference in one or multiple measured parameters.

In some aspects, the techniques described herein relate to a system for characterizing a vasodilation response in a person, the system including: a wearable device configured to be positioned on or adjacent to a lower extremity of a person and to monitor a plurality of skin surface sites of the person, the wearable device including: a stimulus source having a surface area to provide stimulus, and a blood volume sensor configured to sense a vasodilation response of at least one of the plurality of skin surface sites; an electrode configured to obtain bioelectrical data, the electrode being placed in contact with at least one site of the plurality of skin surface sites; and at least one processor communicatively coupled to the wearable device and configured to: output a first activation signal to the stimulus source, output a second activation signal to the blood volume sensor once the at least one of the plurality of skin surface sites reaches a target offset temperature from a baseline temperature, receive an output signal from the blood volume sensor, and determine a vasodilation response of the at least one of the plurality of skin surface sites based on the output signal.

In some aspects, the techniques described herein relate to a system, wherein the lower extremity is an ankle of the person. In some aspects, the techniques described herein relate to a system, wherein the stimulus source is a thermal stimulus source and the lower extremity is a foot of the person, such that at least the thermal stimulus source and the sensor are configured to be positioned on a sole of the foot. In some aspects, the techniques described herein relate to a system, wherein at least the stimulus source and the sensor are configured to be positioned on one of: an arch, a metatarsal region, or a heel of the foot. In some aspects, the techniques described herein relate to a system, wherein the electrode is configured to be positioned on the sole of the foot. In some aspects, the techniques described herein relate to a system, wherein the electrode is configured to be positioned on one of: an arch, a metatarsal region, or a heel of the foot.

In some aspects, the techniques described herein relate to a system, wherein the electrode is positioned on a dorsal region of the foot. In some aspects, the techniques described herein relate to a system, wherein the wearable device is embedded in a shoe insert. In some aspects, the techniques described herein relate to a system, wherein the shoe insert includes silicone, elastane, or a combination thereof. In some aspects, the techniques described herein relate to a system, wherein the shoe insert is porous.

In some aspects, the techniques described herein relate to a system, wherein the lower extremity is a foot of the person, such that at least the stimulus source and the sensor are configured to be positioned on a dorsal of the foot. In some aspects, the techniques described herein relate to a system, wherein the electrode is positioned on a sole of the foot. In some aspects, the techniques described herein relate to a system, wherein the wearable device is integrated into a tongue of a shoe. In some aspects, the techniques described herein relate to a system, further including a band, such that the wearable device is secured to the band and the band is configured to wrap around the lower extremity of the person.

In some aspects, the techniques described herein relate to a system, wherein the band is one or more of: adjustable, stretchable, or tensionable. In some aspects, the techniques described herein relate to a system, further including a patch, such that the wearable device is integrated into the patch and the patch is configured to be secured to the lower extremity of the person. In some aspects, the techniques described herein relate to a system, wherein the patch is configured to be secured to a sole of a foot of the person. In some aspects, the techniques described herein relate to a system, wherein the wearable device is configured to be positioned on one of: an arch, a metatarsal region, or a heel of the foot.

In some aspects, the techniques described herein relate to a system, wherein the patch includes one or more of: a hook and loop fastener or an adhesive. In some aspects, the techniques described herein relate to a system, wherein the wearable device includes a plurality of sensors. In some aspects, the techniques described herein relate to a system, wherein the plurality of sensors include one or more of: a pressure sensor, a motion sensor, a skin temperature sensor, a blood volume sensor, a muscle activity sensor, a sweat sensor, a tissue impedance sensor, or an environmental temperature sensor.

In some aspects, the techniques described herein relate to a system, further including a platform, such that the wearable device is integrated into the platform and configured to be positioned in adjacent to the lower extremity when the person interacts with the platform. In some aspects, the techniques described herein relate to a system, wherein the platform further includes a weight sensor. In some aspects, the techniques described herein relate to a system, wherein the electrode is configured to detect electrodermal activity. In some aspects, the techniques described herein relate to a system, further including a plurality of electrodes. In some aspects, the techniques described herein relate to a system, wherein the plurality of electrodes includes one or more of: a bioimpedance electrode, an electrodermal activity electrode, an electromyography electrode, or an electrocardiogram electrode.

In some aspects, the techniques described herein relate to a system for characterizing a vasodilation response in a person, the system including: a wearable device configured to be positioned on or adjacent to a lower extremity of a person and to monitor a plurality of skin surface sites of the person, the wearable device including: an electrode configured to obtain bioelectrical data, the electrode being placed in contact with at least one site in the plurality of skin surface sites; a blood volume sensor configured to sense a vasodilation response of at least one of the plurality of skin surface sites; and at least one processor communicatively coupled to the wearable device and configured to: output a first activation signal to the blood volume sensor based on a first set of predefined parameters, output a second activation signal to the electrode based on a second set of predefined parameters, receive a first output signal from the blood volume sensor and a second output signal from the electrode, and determine a vasodilation response of the at least one of the plurality of skin surface sites based on the first output signal and the second output signal.

In some aspects, the techniques described herein relate to a system, wherein the lower extremity is an ankle of the person. In some aspects, the techniques described herein relate to a system, wherein the lower extremity is a foot of the person, such that the sensor and the electrode are configured to be positioned on a sole of the foot. In some aspects, the techniques described herein relate to a system, wherein the sensor is configured to be positioned on one of: an arch, a metatarsal region, or a heel of the foot.

In some aspects, the techniques described herein relate to a system, wherein the electrode is configured to be positioned on the sole of the foot. In some aspects, the techniques described herein relate to a system, wherein the electrode is configured to be positioned on one of: an arch, a metatarsal region, or a heel of the foot. In some aspects, the techniques described herein relate to a system, wherein the electrode is positioned on a dorsal region of the foot. In some aspects, the techniques described herein relate to a system, wherein the wearable device is embedded in a shoe insert.

In some aspects, the techniques described herein relate to a system, wherein the shoe insert includes silicone, elastane, or a combination thereof. In some aspects, the techniques described herein relate to a system, wherein the shoe insert is porous. In some aspects, the techniques described herein relate to a system, wherein the lower extremity is a foot of the person, such that the sensor and the electrode are configured to be positioned on a dorsal region of the foot.

In some aspects, the techniques described herein relate to a system, wherein the electrode is positioned on a sole of the foot. In some aspects, the techniques described herein relate to a system, wherein the wearable device is integrated into a tongue of a shoe. In some aspects, the techniques described herein relate to a system, further including a band, such that the wearable device is secured to the band and the band is configured to wrap around the lower extremity of the person.

In some aspects, the techniques described herein relate to a system, wherein the band is one or more of: adjustable, stretchable, or tensionable. In some aspects, the techniques described herein relate to a system, further including a patch, such that the wearable device is integrated into the patch and the patch is configured to be secured to the lower extremity of the person. In some aspects, the techniques described herein relate to a system, wherein the patch is configured to be secured to a sole of a foot of the person. In some aspects, the techniques described herein relate to a system, wherein the wearable device is configured to be positioned on one of: an arch, a metatarsal region, or a heel of the foot.

In some aspects, the techniques described herein relate to a system, wherein the patch includes one or more of: a hook and loop fastener or an adhesive. In some aspects, the techniques described herein relate to a system, further including a platform, such that the wearable device is integrated into the platform and configured to be positioned in adjacent to the lower extremity when the person interacts with the platform. In some aspects, the techniques described herein relate to a system, wherein the platform further includes a weight sensor.

In some aspects, the techniques described herein relate to a method for vasodilation monitoring. The method can include positioning a wearable device against at least one of a plurality of skin surface sites of a person proximate to a foot of the person; outputting a first activation signal to an electronic stimulus source; outputting a second activation signal to a blood volume sensor once the at least one of the plurality of skin surface sites reaches a target offset temperature from a baseline temperature; receiving an output signal from the blood volume sensor; and determining a vasodilation response of the at least of the plurality of skin surface sites based on the output signal.

In some aspects, the techniques described herein relate to a method for detecting vasodilation in a person. The method can include positioning a wearable device against at least one of a plurality of skin surface sites of a person's foot; outputting a first activation signal to a blood volume sensor based on a first set of predefined parameters; outputting a second activation signal to an electrode based on a second set of predefined parameters; receiving a first output signal from the blood volume sensor and a second output signal from the electrode; and determining a vasodilation response of the at least of the plurality of skin surface sites based on the first output signal and the second output signal.

In some aspects, the techniques described herein relate to a wearable device for detecting vasodilation in a person. The wearable device can include a first body configured to be positioned against a plurality of skin surface sites of the person, the first body having a first surface opposite a second surface, wherein the second surface is configured to be in contact with a skin surface of the person; a stimulus source; and a blood volume sensor configured to sense a vasodilation response of at least one of the plurality of skin surface sites, wherein the blood volume sensor and the stimulus source are positioned on the second surface; a second body positioned away from the stimulus source, the second body including at least one electrode; a band such that the wearable device is secured to the band and the band is configured to wrap around a proximity of the person's foot; and at least one processor communicatively coupled to the wearable device, wherein the processor is configured to: output a first activation signal to the blood volume sensor; output a second activation signal to the electrode; receive the first activation signal from the blood volume sensor; receive the second activation signal from the electrode; and determine a vasodilation response based on the first output and the second output signal.

In some aspects, the techniques described herein relate to a wearable device, further including a cutout in the first surface, wherein the cutout includes an inner perimeter, and the cutout is enclosed entirely within an outer perimeter on the second surface.

In some aspects, the techniques described herein relate to a wearable device, wherein the blood volume sensor is positioned within an inner perimeter of the cutout on the first surface.

In some aspects, the techniques described herein relate to a wearable device, wherein the second body includes one or more of a bioimpedance electrode, an electrodermal activity electrode, an electromyography electrode, or an electrocardiogram electrode.

In some aspects, the techniques described herein relate to a wearable device, further including a band such that the wearable device is secured to the band and the band is configured to wrap around the lower extremity of the person.

In some aspects, the techniques described herein relate to a wearable device, wherein the lower extremity of the person is a foot of the person such that at least one of the first body or the second body contacts a sole of the foot.

In some aspects, the techniques described herein relate to a wearable device, wherein the second body is coupled to and integrated with the band.

In some aspects, the techniques described herein relate to a wearable device for detecting vasodilation in a person, the wearable device including: a first body configured to be positioned against a plurality of skin surface sites of the person, the first body having a first surface opposite a second surface, wherein the second surface is configured to be in contact with a skin surface of the person, the first body including: a stimulus source; and a blood volume sensor configured to sense a vasodilation response of at least one of the plurality of skin surface sites, wherein the blood volume sensor and the stimulus source are positioned on the second surface; a second body positioned away from the stimulus source, the second body including at least one electrode; a band such that the wearable device is secured to the band and the band is configured to wrap around a proximity of a foot of the person; at least one processor communicatively coupled to the wearable device, wherein the processor is configured to: output a first activation signal to the blood volume sensor; output a second activation signal to the electrode; receive the first activation signal from the blood volume sensor; receive the second activation signal from the electrode; and determine a vasodilation response based on the first output and the second output signal.

In some aspects, the techniques described herein relate to a wearable device, further including a cutout in the first surface, wherein the cutout includes an inner perimeter, and the cutout is enclosed entirely within an outer perimeter on the second surface.

In some aspects, the techniques described herein relate to a wearable device, wherein the blood volume sensor is positioned within an inner perimeter of the cutout on the first surface.

In some aspects, the techniques described herein relate to a wearable device, wherein the second body includes one or more of a bioimpedance electrode, an electrodermal activity electrode, an electromyography electrode, or an electrocardiogram electrode.

In some aspects, the techniques described herein relate to a wearable device, wherein the at least one of the first body or the second body contacts a sole of the foot.

In some aspects, the techniques described herein relate to a wearable device, wherein the second body is coupled to and integrated with the band.

In some aspects, the techniques described herein relate to a system configured to detect a stroke event based on a measurement of one or more physiological parameters from a sole of a foot, the system including: a physiological sensor including: a blood volume sensor; a shoe insert or a sock configured to integrate a stimulus source and the physiological sensor, wherein the shoe insert or the sock is configured to position the physiological sensor for measurement of the one or more physiological parameters from the sole of the foot of a person; and one or more hardware processors communicatively coupled to the physiological sensor, wherein the one or more hardware processors are configured to: output a first activation signal to the stimulus source configured to heat the sole of the foot; measure one or more physiological parameters from the blood volume sensor responsive to heating the sole of the foot; and determine a stroke event based at least on the measurement of the one or more physiological parameters.

In some aspects, the techniques described herein relate to a system, further including a heater trace.

In some aspects, the techniques described herein relate to a system, wherein the heater trace covers a substantial portion of the sole of the foot.

In some aspects, the techniques described herein relate to a system, wherein a heater trace covers a top and bottom portion of the foot.

In some aspects, the techniques described herein relate to a system, wherein the heater trace includes an opening configured to house physiological sensor.

In some aspects, the techniques described herein relate to a system, wherein the heater trace covers a substantial portion of the sole of the foot except for the opening.

In some aspects, the techniques described herein relate to a system, wherein the heater trace is positioned in a proximity of an ankle of the person.

In some aspects, the techniques described herein relate to a system, wherein the physiological sensor is a temperature senor.

In some aspects, the techniques described herein relate to a system, further including a batter positioned in a proximity of an ankle of the person.

In some aspects, the techniques described herein relate to a system, wherein the physiological sensor is positioned symmetrical along a longitudinal axis of the foot.

In some aspects, the techniques described herein relate to a system, wherein the sensor is symmetrical along a transverse axis of the foot.

In some aspects, the techniques described herein relate to a system, wherein the physiological sensor is intertwined with a fabric of the sock.

In some aspects, the techniques described herein relate to a system, wherein the physiological sensor is sewn to the sock.

In some aspects, the techniques described herein relate to a system for measuring a stroke event in a person from a site of a foot, the system including: a body portion configured to be positioned on a shoe and to monitor a plurality of skin surface sites of the person; a stimulus source having a surface area to provide stimulus; a physiological sensor including a blood volume sensor; a heat source configured to be positioned below a sole of the person; and at least one processor communicatively coupled to the body portion and configured to: output a first activation signal to the stimulus source; output a second activation signal to the blood volume sensor once the at least one of the plurality of skin surface sites reaches a target offset temperature from a baseline temperature; receive an output signal from the blood volume sensor; and determine a stroke event based at least on the output signal.

In some aspects, the techniques described herein relate to a system, wherein the body portion is positioned on a top portion of the shoe.

In some aspects, the techniques described herein relate to a system, wherein the heat source includes heater trace.

In some aspects, the techniques described herein relate to a system, wherein a substantial portion of the sole of the person contacts the heater trace.

In some aspects, the techniques described herein relate to a system, wherein the heater trace is integrated with a shoe insert.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various implementations, with reference made to the accompanying drawings.

FIG. 1A illustrates an example of a wearable device for monitoring physiological parameters of a user.

FIG. 1B illustrates a block diagram of an example physiological monitoring system for obtaining and processing biological data.

FIG. 1C illustrates a block diagram of an example computing environment for processing biological data.

FIG. 2 illustrates a flow diagram of an example process for generating biological data characterizations and/or estimates.

FIG. 3 illustrates a first and second wearable device for measuring biological data and response asymmetry across a right and left limb, respectively.

FIG. 4A illustrates a perspective view of one implementation of a wearable device for obtaining biological data.

FIG. 4B illustrates a perspective view of the wearable device of FIG. 4A.

FIG. 4C illustrates a partial view of the wearable device of FIG. 4A.

FIG. 4D illustrates a rotated view of the wearable device of FIG. 4C.

FIG. 4E illustrates a perspective view and partial exploded view of the wearable device of FIG. 4C.

FIG. 4F illustrates a front view of the wearable device of FIG. 4C.

FIG. 5A illustrates a top perspective view of a wearable device for measuring biological data on a lower extremity.

FIG. 5B illustrates a bottom perspective view of the wearable device of FIG. 5A.

FIGS. 6A-6E illustrate examples of wearable devices positioned on various locations of a lower extremity to measure biological data.

FIG. 7 illustrates another embodiment of a first and second wearable device positioned on the feet to measure biological data.

FIG. 8 illustrates yet another embodiment of a first and second wearable device positioned on the feet to measure biological data.

FIG. 9 illustrates the first and second wearable device of FIG. 8 positioned in another orientation and location on a left foot and a right foot, respectively.

FIG. 10A illustrates an embodiment of a wearable device positioned on an inner portion of an ankle.

FIG. 10B illustrates an embodiment of a wearable device positioned on an outer portion of an ankle.

FIG. 11 illustrates another embodiment of a wearable device positioned on the top or a dorsal region of the foot adjacent to a shoe top.

FIGS. 12A-12B illustrate another embodiment of a wearable device configured as part of a shoe insert.

FIGS. 13A-13B illustrate the devices of FIGS. 5A-5B strapped to a top portion or dorsal region of a left foot and a top portion or a dorsal region of a right foot, respectively.

FIG. 14 illustrates another embodiment of a wearable device configured as part of a platform.

FIG. 15 illustrates a flow diagram of an example process for characterizing and/or monitoring a vasodilation response.

FIG. 16 illustrates a flow diagram of an example process for characterizing and/or monitoring a vasodilation response.

FIGS. 17A-B illustrate another embodiment of a wearable device configured as a part of a sock.

FIGS. 18A-18B illustrate another embodiment of a wearable device configured as a part of a shoe.

The illustrated implementations are merely examples and are not intended to limit the disclosure. The schematics are drawn to illustrate features and concepts and are not necessarily drawn to scale.

### I. DETAILED DESCRIPTION

Disclosed herein are systems and methods for obtaining, monitoring and/or characterizing a vasodilation response of a user. For example, the systems may include wearable devices that can stimulate a skin surface site to obtain the vasodilation response of a user wearing such devices. PCT Application PCT/US2022/071701 filed on Apr. 13, 2022 (incorporated herein in its entirety), including FIGS. 67A through 86B therein, shows example wearable systems and methods for measuring vasodilatory response.

In some implementations, it may be advantageous to position the wearable devices of PCT Application PCT/US2022/071701 (as incorporated above) and additional embodiments described herein on a lower extremity of a person. For example, the wearable devices may be secured to the lower extremity by a band, as described elsewhere herein, via a patch, or the like. The lower extremity may be a shin of the person, an ankle of the person, a foot of the person, or the like. In some implementations, the wearable devices are positioned on a bottom part or sole of a foot where the skin may be more responsive to physiological measurements. This may improve anomaly detection because it was observed by the inventors that physiological signal measurement differences may be larger when measured at skin sites such as bottom of the feet where the blood vessels are closer to the skin. For example, the skin on the sole of the foot (i.e., Glabrous skin) is physiologically similar to that of the palm of the hand, each of which include sweat glands that are more reactive to psychogenic stimuli (e.g., emotions). The responses may be measurable using electrodermal activity (EDA) sensing. Furthermore, through physiology of the eccrine sweat gland innervations of the glabrous skin, EDA is considered to be a direct measure of sympathetic outflow. Assessing the asymmetry in sympathetic outflow, as measured by EDA, may provide an improved analysis and/or assessment (e.g., detection) of asymmetrical differences between two extremities (or between two extremity portions), which may help to indicate unilateral brain lesions such as those during stroke or neurological decline.

Additional improvements in some instances can include reduction in noise or artifacts in a sensor signal retrieved from wearable devices positioned at lower extremity. The noise and/or artifacts may be reduced because there may be more time periods of reduced motion in the lower extremities of an awake person than would be present in measurements obtained from the upper extremities of an awake person. For example, a person often uses their hands while talking, which impacts signal quality, but the feet remain still. Additionally, raw signal quality may be improved when obtained from the lower extremities than at other locations of the body, for example because of the reduced fat content of the lower extremities and capillary density of the feet. Furthermore, the lower extremities may be more likely to experience larger temperature swings than upper extremities, for example, because lower extremities are further away from the core (e.g., heart) of the person. Accordingly, devices positioned on one or more lower extremities may lead to capturing temperatures and anomalies with larger ranges than can be captured on upper extremities. This larger range of temperatures, for example, may be used to determine whether a person is experiencing edema and/or other changes that may cause a large swing in temperature, EDA, heart rate, etc.

As discussed earlier, in some implementations, the wearable devices are positioned on a bottom part or sole of a foot. A foot of a user or a patient can have a larger area than their wrist. This provides a foot-worn device with more surface area to use and collect data from, which can help with measuring physiological parameters more accurately. For example, more sensors can spread over a larger area whereas doing so on the wrist could result in a bulky arrangement. Similarly, a larger surface area can be allocated to different components. For example, a large surface area over a sole of a foot can be allocated to a heating source whereas in a wrist-worn device, this surface area is limited to the available area on the user's wrist. Furthermore, the available area on a person's wrist may be limited by other devices or components that the user may wear. For example, a user may want to wear a bracelet, a watch, a wristband etc. These articles can occupy a larger area of the user's wrist and further limit the available area for another device. From an aesthetic standpoint, the user may not want the wearable device to be visible to public. A foot-worn wearable device can enable the user to have their wearable device(s) covered by other articles (such as their shoes, pants, socks, etc.). Therefore, allowing a user to utilize a foot-worn device to measure their physiological parameters can provide users/patients a better experience for measuring physiological parameters and/or their health data.

### Wearable Physiological Monitoring Device

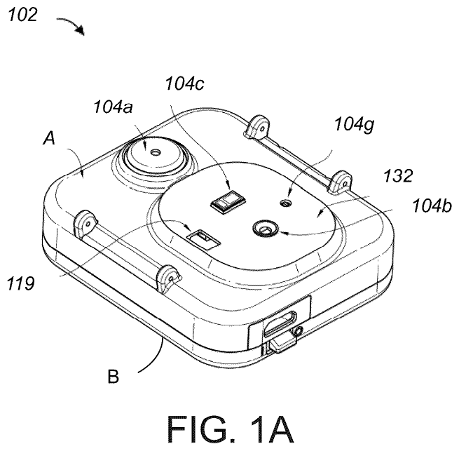

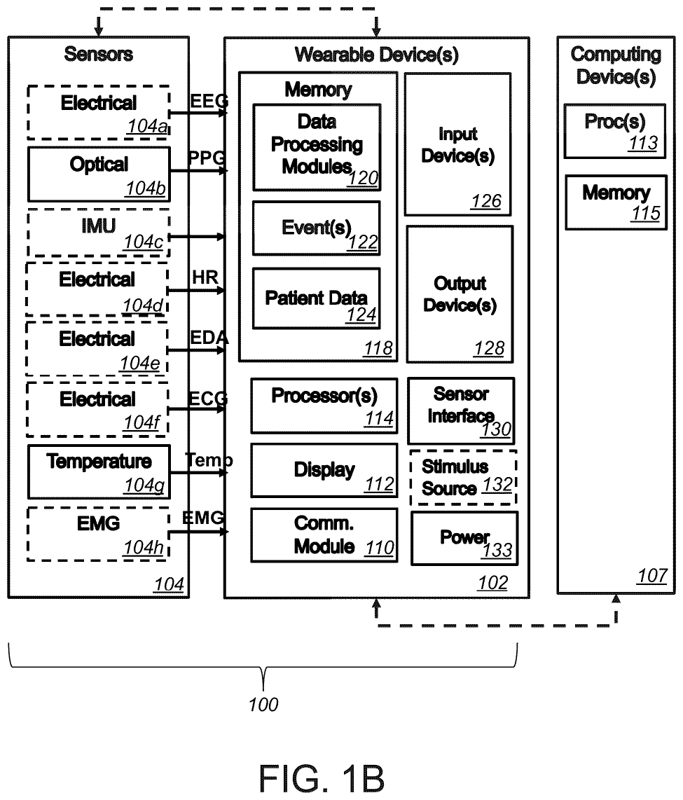

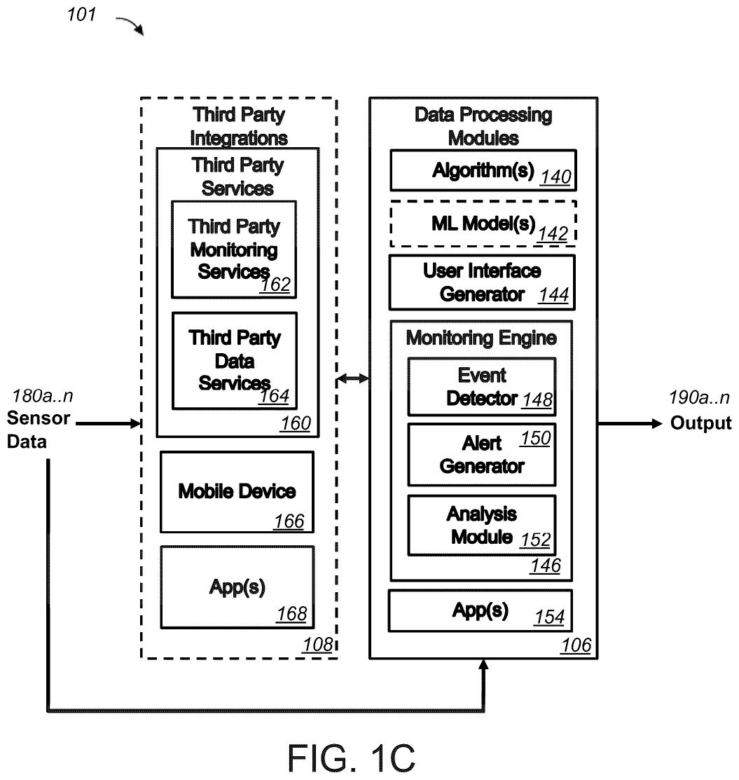

FIG. 1A illustrates an example of a wearable device 102 for monitoring physiological parameters of a user (e.g., a person, a patient). The wearable device 102 can include one or more sensors for detecting physiological parameters of the user. For example, the wearable device 102 can include a sensor 104a (e.g., an electrical sensor) for obtaining temperature responses and/or EDA responses from a skin surface site of the user. The wearable device 102 can also include an optical sensor 104b for obtaining a temperature and/or a photoplethysmographic (PPG) measurement from a skin surface site of the user. Other sensors and/or elements may be incorporated into or near to the device 102, as will be described in detail below.

The wearable device 102 may be configured to measure any number of physiological parameters of a user wearing the device 102. The measurements may be retrieved using one or more sensors integrated into or associated with device 102. For example, the device 102 may include one or more sensors for measuring core and/or skin surface temperatures; volumetric impedance spectroscopy; hyperhidrosis; heart rate or heart rate variability; and/or motion (e.g., by including an accelerometer and/or gyroscope therein) to measure, for example, limb asymmetry or changes in gait. In particular, device 102 may include one or more of a pressure sensor, a motion sensor, a skin temperature sensor, a blood volume sensor, a muscle activity sensor, a sweat sensor, a tissue impedance sensor, or an environmental temperature sensor. In addition, the device 102 may be communicatively coupled (e.g., via antenna, coils, etc.) to an external sensor that is not housed in or integrated into device 102.

Some or all components of the wearable device 102 may be integrated into a patch, a band, a watch, an adhesive strip, a bracelet, an anklet, a sock, a shoe insole, a shoe, clothing, or any other wearable accessory. For example, some or all components of the wearable device 102 may be incorporated into a ring or band or a pair of rings or bands to be worn one on each ankle or each foot. In some implementations, the ring or band may incorporate a stretchable or expandable element or stretch sensor to allow the ring or band to expand or stretch when the foot, ankle, toe, etc. swells. This element may include, but is not limited to, elastomer film polymers of various degree of bonding to allow for different pliable elements or measuring the reflectivity of polarized light. This element may include a plastic segment of the ring or band that can be loosened/tightened, or by building a slidable element that can be pulled apart. Non-limiting examples of a stretch sensor include, but are not limited to, a strain gauge or an electrical component which can change inductance, resistance, or capacitance when stretched.

Referring again to FIG. 1A, the wearable device 102 includes a body portion with a first surface A opposite a second surface B. The first surface A may be placed in contact with a skin surface of a user to obtain measurements and provide a stimulus via a stimulus source 132, for example heat or coolness via a thermal stimulus source. The first surface A and second surface B may be coupled via one or more or a plurality of sidewalls of device 102. For example, one or more sidewalls 405 may extend from a perimeter of the first surface A and couple to a perimeter of the second surface B. The first surface A and/or second surface B may include one or more sensors 104 (e.g., sensor or electrode 104a, sensors 104b, 104c, 104d, 104e, 104f, 104g, and/or 104h, etc. as shown in FIGS. 1A-1B) positioned thereon. For example, one or more sensors 104 on the first surface A may measure an environment of the user wearing or using the wearable device 102, and one or more sensors on the second surface B may measure one or more properties, features, or characteristics of the skin surface of the user. In some implementations, the wearable device 102 may include an opening, such as opening 119, to allow the heat source 132 to communicate with and/or access other components inside the body of device 102, for example processor(s) 114 and/or power source 133.

The wearable device 102 may be worn on an exterior or skin surface of a user (e.g., a patient). In some implementations, the wearable device 102 may include a strip or form a strip that measures muscle contractions through surface electromyography (sEMG). The measurement of EMG may be compared to a baseline value to detect a change or asymmetry of the EMG. In some implementations, EMG measures a user's intent to move a muscle and a corresponding actual user movement of the same muscle, compared to a typical movement profile of the muscle or a movement response on an opposite limb or extremity.

In some implementations of device 102, one or more sensors/electrodes 104a may be integrated into a body of the wearable device 102 or in a separate component of the wearable device 102. Further, in some implementations of the device 102, a stimulus source 132 is not utilized when monitoring and/or characterizing a vasodilation response. In a non-limiting example of a wearable device without a stimulus source 132, fluid shift within the vasculature (e.g., when someone moves from laying to sitting to standing) may be sufficient to detect vasodilation as a result of the fluid shift. For example, the wearable device 102 may detect limb position or perform pulse wave analysis, to evaluate aspects of vasodilation. In one exemplary, non-limiting embodiment, when the arm is up in the air, the blood drains out of the hand resulting in the photoplethysmogram alternating current (AC) amplitude dropping. With the arm down by the side, the AC amplitude may increase because of blood pooling in the hand. The same effects occur in the feet when moving between standing, sitting, and laying down due to gravity induced blood pooling. A communicatively coupled accelerometer or an accelerometer embedded in the wearable device 102 may be used to detect limb position so that limb position may be tracked and a blood volume signal recorded based on the detected position.

In some implementations, the stimulus source 132 is not included in the wearable device 102. Instead, ambient environment temperature fluctuations that naturally alter vasodilation at one or more skin surface sites may be used to detect vasodilation. An ambient temperature fluctuation may include hot or cold water, under the covers, natural sunlight, warm or cold outdoors, and the like. Alternatively, the stimulus source 132 may be an external stimulus source, for example a laser, a hot or cold environment, or hot or cold objects placed near to the device. For example, when the wearable device 102 does not include a stimulus source 132, a processor of the wearable device 102 may receive a first skin temperature at time A and may receive a second skin temperature at time B to compare the underlying state of the blood vessel vasodilation or vasoconstriction. For example, the underlying state of a blood vessel may refer to a change in a diameter of the blood vessel and/or how the diameter of the blood vessel changes over time due to vasodilation and/or vasoconstriction. Internal studies show that core body heating due to exercise increases vasodilation without any external heating stimulus. In such instances, blood volume measurements may be substantially continuous rather than a stimulus-based response.

In some implementations, the wearable device 102 may generate a stimulus for delivery to a monitoring site and measure a response in physiological parameters based on the stimulus. The stimulus may be applied to one location or a plurality of locations. In one implementation, the stimulus is applied bilaterally (e.g., to detect asymmetrical responses) on the body of the user to determine whether the response or the difference in response between the two sides indicates an atypical event such as a stroke event, neurological decline, or otherwise a deviation from baseline. For example, the stimulus may be applied in a stimulus cycle such that the baseline, during stimulation, and post stimulation responses are measured, or change in (e.g., slope, decay, etc.) responses between different measurement periods are determined. For example, a thermal (i.e., hot or cold) stimulus may be applied to a section of skin on a body of a user (shown in top panel) and the body's response to the thermal stimulus may be monitored over time (shown in bottom panel) to determine whether homeostasis is reached and/or a difference in response or return rate exists between the two sides of the body (in other words, determine whether an asymmetrical response exists). Responses can be indicative of changes in or perturbations in the parasympathetic nervous system, the sympathetic nervous system, the central vascular system, or the peripheral vascular system.

Further examples include stimulating the muscular or nervous system using electrical signals and monitoring the response over time and/or between sides using electromyogram (EMG), bioimpedance, or electroneurogram (ENG), respectively. These “stimulators/transmitters” and “receivers/detectors” could be in the same region or could be separated to measure across regions of the body.

Applying heat stress (e.g., stimulation) to a portion of the skin may enable detection of a vasodilation response. In some implementations, the wearable device 102 may function to heat the portion of the skin (e.g., a skin surface site) and may measure a vasodilation response of the portion of the skin. The device 102 may further function to measure one or more additional parameters, biologic signals, etc. as will be described in greater detail elsewhere herein. In some implementations, the device 102 may use a measured bioimpedance (BioZ) to validate or invalidate a vasodilation measurement from another sensor on device 102.

In some implementations, the device 102 may include and/or communicate with a device positionable in a room, office, home, vehicle, or other location; or in or on a bed or other furniture (e.g., bedside monitors; monitors within mattresses, bedding, etc.). For example, a smart speaker (e.g., to prompt a user to respond to a question to analyze speech quality), microphone, camera, and/or mirror may be positionable in a location to detect changes in a user's speech, activities, movement, gait, facial appearance, heart rate, and/or heart rate variability or changes from baseline. The device may include one or more data processing modules to differentiate changes in the measured parameters as compared to that from healthy learned patient data or individualized baseline data.

The wearable device 102 may be worn on an exterior or skin surface of a user (e.g., a patient) prior to, during, and/or after an anomalous biologic event, including up to days before the event, during the event, and/or after the event to provide continuous variable monitoring of various physiological parameters. For example, the wearable device 102 may function to monitor or characterize a vasodilation response in response to application of a stimulus (heating or cooling) or as a result of fluctuations in vasodilation (without a stimulus source).

### Physiological Monitoring System

FIG. 1B illustrates a block diagram of a physiological monitoring system 100. The system 100 can include a device 102 as described above and one more sensors 104. The system 100 may measure, characterize, or detect physical responses (e.g., stroke events, neurological decline, stress response, heat stroke, seizure, menopause, diabetes, etc.) for a user wearing a wearable device that includes (or is in communication with) monitoring system 100. In some implementations, the sensors 104 can be integrated with the device 102. In additional implementations, some or all of the sensors 104 may be physically separate from the device 102. The sensors 104 can be communicatively coupled to the wearable device 102 including wired and/or wireless connections. The system 100 may also connect with a computing device 107 as described below. Alternatively, or additionally, the system 100 may integrate with third-party devices and/or services.

In some implementations, the wearable device 102 can include a communication module 110, a display 112, processors 114, and memory 118. The display 112 may not be included in all implementations. The communication module 110 may include one or more antennas or coils for wireless connections. The communication module 110 may also include components for wired connection, such as USB data transfer.

The processors 114 may include one or more hardware processors, including microcontrollers, digital signal processors, application specific integrated circuit (ASIC), a field programmable gate array (FPGA) or other programmable logic device, discrete gate or transistor logic, discrete hardware components, or any combination thereof designed to perform the functions described herein and/or capable of executing instructions, such as instructions stored by the memory 118. The processors 114 may also be able to execute instructions for performing communications amongst wearable device 102, sensors 104, data processing modules 106, and/or third-party integration 108.

The memory 118 can include one or more non-transitory computer-readable storage media. The memory 118 may store instructions and data that are usable in combination with processors 114 to execute algorithms 140, optional machine learning models 142, monitoring engine 146 tasks, and apps 154. The memory 118 may also function to store or have access to the data processing modules 120, events 122, and patient data 124.

The system 100 may further include or be communicatively coupled to input devices 126, output devices 128, sensor interface 130, stimulus source 132, and/or power (e.g., battery) 133. The input devices 126 may interact with one or more processors 114, memory 118, and/or sensors 104. The input devices 126 may include buttons, touchscreens, switches, toggles, and/or other hardware components located on wearable device 102. In some implementations, the input device 126, or at least some functionality of input device 126, may be external to or not integrated into the device 102, such that one or more controllers, mobile device (e.g., mobile device 166), apps (e.g., apps 168), etc., may communicate (e.g., antenna, coils, etc.) with device 102 using a wireless communications protocol. In some implementations, the input device 126 may include, for example, a touch input device that can receive tactile user input, a microphone that can receive audible input, and the like.

The output devices 128 may interact with one or more processors 114, memory 118, and/or sensors 104. The output devices 128 may include, for example, a display (e.g., display 112) for visual output, a speaker for audio output, and/or haptic feedback.

The sensor interface 130 may store instructions to carry out operations pertaining to received sensor signals from one or more of the sensors 104. For example, the instructions may enable interaction with one or more processors 114, memory 118, and/or sensors 104 to communicate sensor data from one or more of the sensors 104 to the wearable device 102. The sensor data may be obtained from the sensors 104 taking recordings and measurements. The sensors 104 may include any or all the electrical sensors 104a, 104d, 104e, 104h, optical sensors 104b, 104g, and/or mechanical sensors (e.g., strain gauge, sensor 104c, etc.), as described in detail throughout this disclosure.

The stimulus source 132, in one embodiment, may be a heating element, a thin film resistance flexible heater, a polyimide heater, an optical heater (e.g., a laser), and the like. In other embodiments, the stimulus source 132 may be a cooling element, a thermoelectric cooler, a miniature compressor, or the like. The stimulus source 132 may be placed in communication with the skin surface. The stimulus source 132 may heat or cool the skin surface to a target offset temperature or a pre-determined temperature. In some implementations, the stimulus source 132 may not be integrated into the wearable device and may instead be an environmental heat source, for example a warm room, warm enclosure, and/or other warm environment.

Temperature sensors 104g that may be integrated within and/or communicatively coupled to device 102 may include, but are not limited to, infrared sensors, thermometers, thermistors, or thermal flux transducers. Hyperhidrosis measurement devices may include, but are not limited to, detection of analytes including ions, metabolites, acids, hormones, and small proteins through potentiometry, chronoamperometry, cyclic voltammetry, square wave stripping voltammetry, or detection of changes in conductivity.

The power source 133 may include or connect to a battery or a port for connecting the device 102 to a power supply, a wall power adapter, or the like.

The sensors 104 may be controlled by one or more processors 114. Furthermore, the one or more processors 114 may also obtain and process sensor signal data. Additionally, the one or more processors 114 may communicate raw or processed sensor signal data to third-party integrations 108. The sensors 104 may include one or more of an electroencephalogram (EEG) sensor 104a, a blood volume sensor 104b (e.g., a photoplethysmographic (PPG) sensor), an inertial measurement unit (IMU) sensor 104c, a heart rate (HR) sensor 104d, an electrodermal activity (EDA) sensor 104e, an electrocardiogram (ECG) sensor 104f, a temperature sensor 104g (e.g., a skin temperature sensor), and an electromyography (EMG) sensor 104h and/or mechanical sensors (not shown).

Although various sensor technologies are described elsewhere herein, additional, non-limiting sensors that may be integrated into device 102 or communicatively coupled (e.g., antennas, coils, etc.) to device 102 include: a photoplethysmographic (PPG) device, a skin conductance sensor measuring skin conductance/galvanic skin response (GSR) or electrodermal activity (EDA), or a skin temperature measurement device (e.g., contact devices and non-contact devices, like IR imaging camera).

The EEG sensor 104a may include one or more electrodes that can detect abnormalities in electrical activity of the brain.

The blood volume sensor 104b may include an optical sensor to detect blood volume changes in tissue. The blood volume sensor 104b may measure volumetric impedance spectroscopy, heart rate, respiration rate, or heart rate variability through monitoring a rate of blood flow.

The IMU sensor 104c may monitor motion (e.g., by including an accelerometer and/or gyroscope therein) to measure, for example, limb asymmetry or changes in gait or limb movement.

The heart rate sensor 104d may include one or more electrical sensors (e.g., electrocardiography), optical sensors (e.g., PPG), or mechanical sensors (e.g. accelerometer, strain gauge, pressure sensors) to measure and monitor a heart rate.

The EDA sensor 104e may measure hyperhidrosis. Hyperhidrosis measurement devices may include, but are not limited to, evaluating sweat rate; detection of analytes including ions, metabolites, acids, hormones, and small proteins through potentiometry, chronoamperometry, or cyclic voltammetry; or detection of changes in conductivity.

The ECG sensor 104f may include a plurality of electrodes for recording electrical signals of the heart, to determine heart rate, determine cardiac rhythm, cardiac autonomic control and other cardiovascular and cardiorespiratory metrics. In instances of a stroke event or an event indicating neurological decline, changes in these signals may indicate a potential stroke state or neurological decline.

The temperature sensor 104g may measure temperature. Temperature sensors may include, but are not limited to, infrared sensors, infrared imaging cameras, thermometers, thermistors, or thermal flux transducers. Temperature sensors are used to measure the core body temperature and surface temperatures at various sites on the body. In a stroke event, or a neurological decline event, there may be variations in temperature related to disruptions in temperature regulation, at the core and/or at individual surface sites, that may indicate a stroke event.

The EMG sensor 104h may include two or more or a plurality of electrodes for recording electrical signals of muscles. For example, an EMG sensor 104h may be used to measure an intent to move signal and a resulting movement signal of a muscle. In instances of a stroke event, or a neurological decline event, the intent to move signal may be recorded but the movement signal may be absent, indicating a potential stroke state or an event indicating neurological decline.

Additional metrics that may be obtained by device 102 with one or more additional sensors 104, processors 113 and/or processors 114, and/or electrodes may include, movements, reflexes, stimulus response output, breathing patterns, metrics responsive to audio input, etc.

In some implementations, the wearable device 102 may be communicatively coupled to a computing device 107. The computing device 107 may be a mobile device, a laptop device, a second wearable device, or the like. The computing device 107 may be a computer-executable component that includes processors 113 and memory 115 that may be used to execute the methods described herein. In some implementations, the computing device 107 may be used as a hub to send data to one or more server(s). The computing device 107 and/or particular software application(s) installed on the computing device 107 may also be used to control operation of the wearable device 102.

In some implementations, the system 100 or environment 101 (operating on device 102) may further include a software application downloaded and/or stored on a hardware component of device 102 or on a hardware component of computing device 107. The application may process sensor data, electrode data, camera data, speech data, and/or display data sensed or captured in real time, for example in a graphical representation, of the data.

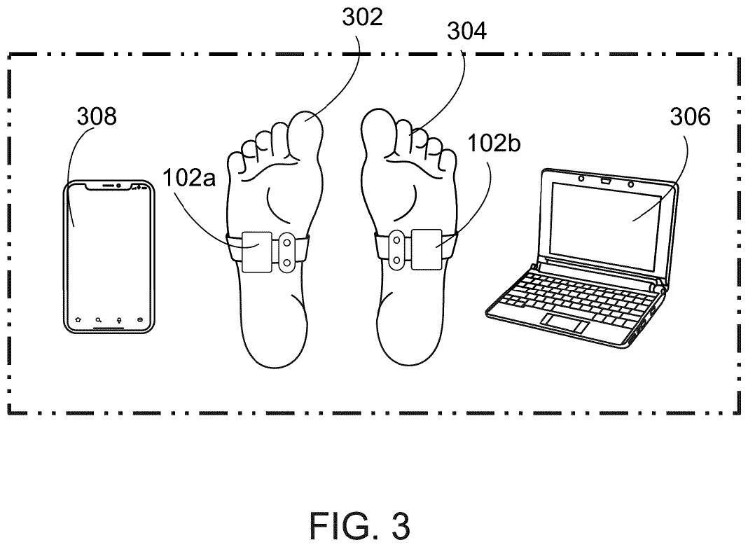

While the system 100 is depicted with a single wearable device 102, any number of wearable devices 102 may be included or communicatively coupled to system 100 (e.g., a first wearable device 102a and/or a second wearable device 102b of FIG. 3). In some implementations, when multiple wearable device(s) are employed, it is not necessary that all wearable devices include all of the components described above. For example, when two wearable devices 102 are used, one of the wearable devices may have limited processing power as all of the processing may be performed on a different wearable device 102. In some instances, one of the wearable devices may include a limited number communication modules.

Data obtained with wearable device 102 may be transmitted to and/or from the system 100 (and/or environment 101 and/or computing device 107) to a central hub, mobile computing device (e.g., computing device 107), server, or other storage and/or computing device. Sharing of such data is performed according to user permissions. For example, a user of device 102 may configure data sharing permissions for device 102 to ensure that the data being shared is shared according to the configured user permissions.

Data transmissions described herein may include wireless communication (e.g., a nearfield communications (NFC) protocol, a low energy Bluetooth® protocol, other radiofrequency (RF) communication protocol, etc.) between sensor locations on the body and/or a central hub. In other implementations, data transmission may include wire communication between sensor locations on the body and/or a central hub. In some implementations, the central hub may be a monitor in a medical facility, home monitor, patients' mobile computing device, or other wireless device. Alternatively, one or more of the sensors on the body may act as the central hub. The hub device may wirelessly send signals to activate a medical care pathway and/or notify one or more individuals (e.g., family, friends, physician, EMS, etc.). In some implementations, data transmission, following multivariate analysis, to the central hub may alert the patient, the next of kin, and/or a third party to identify possible false positives or negatives.

### Processing System(s)

FIG. 1C illustrates a block diagram of an example computing environment 101 for processing biological data. Some or all aspects of the computing environment 101 may be implemented by the systems and devices described above. As shown, the computing environment 101 includes data processing modules 106 and optional (shown by dashed lines) third-party integrations 108. The data processing modules 106 can include multiple engines for performing the processes and functions described herein. The engines can include programmed instructions for performing processes as discussed herein for detection of input conditions and control of output conditions. The engines can be executed by the one or more hardware processors of the device 102 alone or in combination with other hardware devices, such as the computing device 107. The programming instructions can be stored in a memory as discussed above. The programming instructions can be implemented in C, C++, JAVA, or any other suitable programming languages. In some embodiments, some or all of the portions of the data processing modules 106 including the engines can be implemented in application specific circuitry such as ASICs and FPGAs. Some aspects of the functionality of the controller associated with data processing modules 106 can be executed remotely on a server (not shown) over a network. While shown as separate engines, the functionality of the engines as discussed below is not necessarily required to be separated. Accordingly, the data processing modules 106 can be implemented with the hardware components described above with respect to FIGS. 1A-1B.

In operation, the data processing modules 106 and/or the third-party integrations 108 may receive one or more inputs (input sensor data 180a through 180n (e.g., 180a . . . 180n)), and may process the inputs and generate output data 190a through 190n (e.g., 190a . . . 190n). For example, the data processing modules 106 and/or the third-party integrations 108 may receive sensor data from electrical sensor 104a and the optical sensor 104b may process the received data via algorithms 140, ML models 142, and/or monitoring engine 146 to generate a vasodilation estimation (and/or a vasoconstriction estimate) for display on an app 154. To carry out the processing, one or more data processing modules 106 may interact with one or more processors 114, memory 118, and/or sensors 104 to determine a vasodilation response, a vasoconstriction response, and/or related physiological responses. Monitoring and/or characterizing vasodilation responses may result in indications of biological changes, such as menopause, or anomalous biologic events, such as stroke, neurological decline, diabetes, peripheral blood circulation disorders, etc.

As shown in FIG. 1C, the data processing modules 106 include algorithms 140, optional ML models 142, user interface generator 144, monitoring engine 146, and apps 154. The monitoring engine 146 can further include an event detector 148, an alert generator 150, and an analysis module 152. In some implementations, the data processing modules 106 may be stored and executed as data processing modules 120 (FIG. 1B) on wearable device 102.

The algorithms 140 may include computer executable code adapted to carry out any of the methods described herein. In some implementations, the algorithms 140 utilize third party integrations 108 and/or optional ML models 142 to generate output. In operation, the algorithms 140 may operate on particular sensor data 180a . . . 180n (e.g., obtained from sensors 104 on device 102). The operations may prepare the data (e.g., preprocess) and/or otherwise analyze the data to generate estimations (e.g., PPG, respiration rate, heart rate, event occurrence, etc.). The data processing modules 106 can control operations of the device 102. For example, the data processing modules 106 can output a first activation signal to a stimulus source.

The optional ML models 142 may use machine learning techniques to estimate vasodilation activity for blood vessels associated with a particular skin surface site. For example, the ML models 142 may perform analysis, pattern classification, and/or recognition algorithms on PPG signals to assess changing optical properties of underlying tissues triggered by a stimulus provided by a wearable device. Portions of the analyzed signals may be used to generate vasodilation activity estimations. In some implementations, the ML models 142 may operate on signals obtained from sensors 104. The operations may include signal processing that may employ signal processing tools, for example filtering, extracting, digitizing, data de-convolution, machine learning, and/or other methods known in the art. Specifically, the signal processing may use higher order statistics to ascertain hidden patterns in data. Use of higher order statistics, known as cumulants, and their Fourier spectra, often termed poly spectra, not only reveal the amplitude information in the higher order (such as those carried by power spectra or auto correlation) but may also include phase information. Phase information can reveal salient features of the data, otherwise unattainable from simple harmonic analysis.

The user interface generator 144 may interact with one or more processors 113, 114, memory 115, 118, and/or sensors 104. The user interface generator 144 may generate user interfaces for display to a user of the wearable device 102 or other user associated with the wearable device 102. The user interfaces may include patient data, sensor measurements and data, instructions, diagnosis data, or the like. The user interface may be presented on the display 112 of the wearable device and/or on a display of a companion device such as a mobile phone, laptop, tablet, or computer configured to receive information and user interfaces from the wearable device 102. The user interface may be configured to display a vasodilation response of the user over time, relative to a population, across multiple skin sites of a user, etc. The user interface may be further configured to display additional physiological responses of the user over time, relative to a population, across multiple skin sites, etc., for example an electrodermal activity response, muscle activity, hydration state, etc. The vasodilation response and physiological response(s) may be overlaid or displayed independently depending on the intended analysis.

The monitoring engine 146 may determine when events or data changes occur at one or more of the sensors 104 (or associated electrodes). The monitoring engine 146 includes an event detector 148, an alert generator 150, and an analysis module 152. The event detector 148 may monitor and detect biological signals to monitor or characterize the signals or determine whether an anomalous biologic event has occurred. The analysis module 152 may function in combination with the event detector 148 to determine whether an event (or change in biological data) is to trigger an alert. The alert generator 150 may receive the determination of whether to trigger an alert and in response, may trigger the alert. For example, the temperature sensor 104g may function with the monitoring engine 146 to monitor and obtain temperature at a particular skin surface site associated with the wearable device 102. The monitoring engine 146 may trigger the alert generator 150 to generate an alert in response to determining that a monitored temperature has reached or exceeded a predefined threshold temperature.

The alert generator 150 may alert the user wearing the wearable device 102 and/or alert a third party of an event, available data, and/or a change in data. In some implementations, the alert may be an audible sound or a visual indicator or message to the user via the device 102 or via a mobile device, computing device associated with the device 102. In some implementations, the alert may be a message sent to emergency services or physicians. Alerting emergency services or physicians, data including medical history may be transmitted directly to emergency services or physician computing systems, either directly from the wearable device 102 or from a remote memory, initiated by a signal from the wearable device 102. In addition to alerts, the wearable device 102 can also instruct a user to undertake or automatically activate certain treatments.

The apps 154 may represent one or more software applications that may be installed on (or accessed from) the wearable device 102 (and/or an associated mobile phone or computing device 107). The apps 154 may be used to present user interfaces generated by user interface generator 144.

Optionally (shown by dashed lines), the environment 101 may further include a third-party device or integration 108, for example a device including Amazon® Alexa® or an Amazon® Echo® device, as described in further detail elsewhere herein. For example, there may be bidirectional communication (e.g., via a wired connection or wireless communication) between the hardware component and the data processing modules 106, the data processing modules 106 and the third-party device or integration 108, and/or the third-party device or integration 108 and the hardware component.

Third party integrations 108 may optionally function with the wearable device 102, sensors 104, and/or data processing modules 106 to generate additional data for a user or medical practitioner. Third party integrations 108 may include third party services 160, third-party monitoring services 162, third party data services 164, mobile device 166, and apps 168.

Third party services 160 may provide monitoring services 162 and/or data services 164 to device 102 via a Wi-Fi network, a cellular network, cloud networks, or the like. The third-party monitoring services 162 and/or data services 164 may be provided directly to the device 102 or indirectly to device 102 via a mobile device 166. In some implementations, the third-party monitoring services 162 and/or data services 164 may be provided indirectly to the wearable device 102 via apps 168 that may interact with sensor data and device 102.

In some implementations, the data processing modules 106 and algorithms 140 described herein may be executed on wearable device 102. In some implementations, the data processing modules 106 and algorithms 140 described herein may be executed on the system 100. In some implementations, the data processing modules 106 and algorithms 140 described herein may be executed on environment 101. In some implementations, the data processing modules 106 and algorithms 140 described herein may be executed on computing devices (e.g., computing devices 107) or third-party integrations (e.g., third-party integrations 108). In some implementations, the data processing modules 106 and algorithms 140 described herein may be executed in a distributed fashion across one or more of wearable device 102, system 100, environment 101, computing device 107, and/or third-party integrations 108.

Although not depicted in FIGS. 1A-1C, a network may enable communications amongst wearable device 102, sensors 104, data processing modules 106, and/or third-party integrations 108. The network may also be configured through communication module 110 to enable communications with computing devices that are configured to communicate with device 102.

### Example Biological Characterization Process

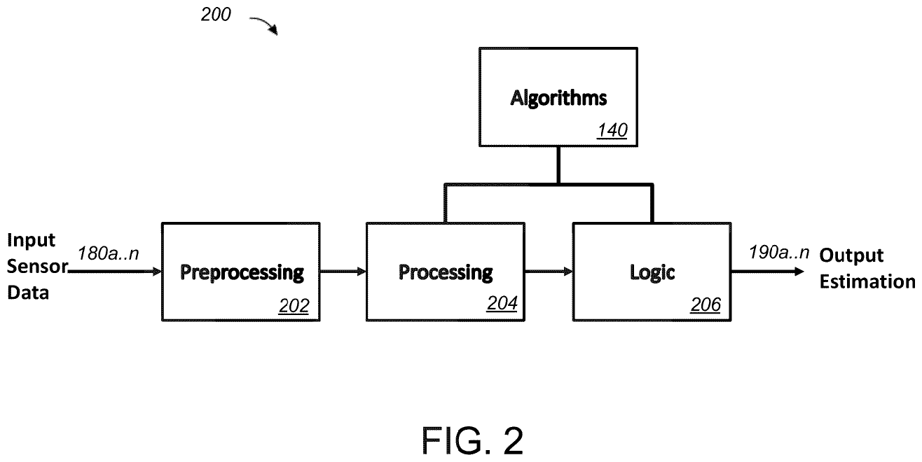

FIG. 2 illustrates a flow diagram of an example process 200 for generating biological data characterizations and/or estimates. The process 200 can be implemented by any of the devices and/or systems discussed above. The process 200 can include the steps of obtaining sensor data corresponding to at least one of inputs 180a through 180n (e.g., 180a . . . 180n), preprocessing (202) at last a portion of the sensor data, processing (204) the preprocessed data, and performing (206) logic to characterize vasodilation of one or more skin surface sites. In some implementations, the processing block 204 and/or logic block 206 may include any or all steps associated with algorithms 140.

For example, an optical sensor input 104b may be received as input 180b into preprocessing block 202. The preprocessing block 202 may perform signal preparation, signal normalization, and/or noise reduction before providing a preprocessed signal to processor block 204. Processing block 204 may use one or more algorithms 140 to perform analysis to generate a vasodilation estimation based on the original signal data 180a . . . n. A processed signal may be provided to logic block 206 to undergo comparisons, and/or further signal analysis. In some implementations, the processing block 204 and the logic block 206 may function to iteratively pass data back and forth to further process the signals and/or related data. A vasodilation estimate may be generated by logic block 206 upon meeting predefined criteria described in detail throughout this disclosure and in at least PCT Application PCT/US2022/071701 (as incorporated above).

### Systems for Characterizing Bilateral Biological Response(s)

FIG. 3 illustrates a first wearable device 102a and a second wearable device 102b for measuring biological data and response asymmetry across a right and left limb, respectively. In this example, the device 102a is worn on a left limb 302 of a body of a person and the device 102b is worn on a right limb 304 of the body of the person. In some implementations, the wearable device 102a is positioned on a left limb or appendage (e.g., arm, leg, finger, hand, foot, toe, ear, etc.) or extremity of a person and a second device 102b is positioned on a right limb or appendage (e.g., arm, leg, finger, hand, foot, toe, ear, etc.) or extremity of the person. The first and second devices 102a, 102b may measure similar parameters or features so that the parameters or features are comparable over time and/or on an event-by-event basis to detect biological signals, asymmetrical biologic responses, and/or deviation from a baseline (e.g., individualized or population based). For example, the data processing modules 106 may compare right side blood volume signals (e.g., in response to an application of heat) to left side blood volume signals (e.g., in response to application of heat) to determine whether an anomalous biologic event has occurred. In some implementations, the application of heat may be applied to a skin surface by both devices 102a, 102b to stimulate or alter biological signals that may later be analyzed to generate predictions, estimates, or treatment recommendations. Further, the stimulus signal delivery between the two devices may be synchronized and accordingly the signals acquired from a right lower extremity and a left lower extremity may also be substantially synchronized in time; and comparing the synchronized signals from the left lower extremity and the right lower extremity to determine whether the anomalous biologic event occurred. Although two devices and body portions are shown, one of skill in the art will appreciate that a single body portion or site may be monitored, characterized, or otherwise analyzed over time. The analysis may be in comparison to a baseline, a population-based level, historical data, or the like.

For example, the wearable device 102a may be applied to a skin surface site on a left lower extremity (e.g., left limb 302) and the wearable device 102b may be applied to a skin surface site on a right lower extremity (e.g., right limb 304). Both devices 102a, 102b may stimulate a response and measure the response from the left and right lower extremities, respectively. For example, the stimulus can be applied bilaterally (e.g., to detect asymmetrical responses) to each device 102a, 102b. The data processing modules 106 may receive signal data detected by sensors of devices 102a, 102b and may determine whether the responses or the difference in responses between the two sides indicates an atypical event, a stroke event, a neurological decline event, or a deviation from baseline.

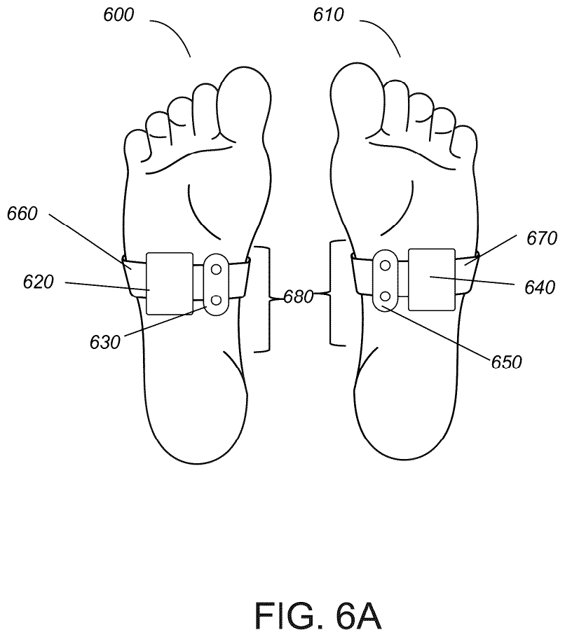

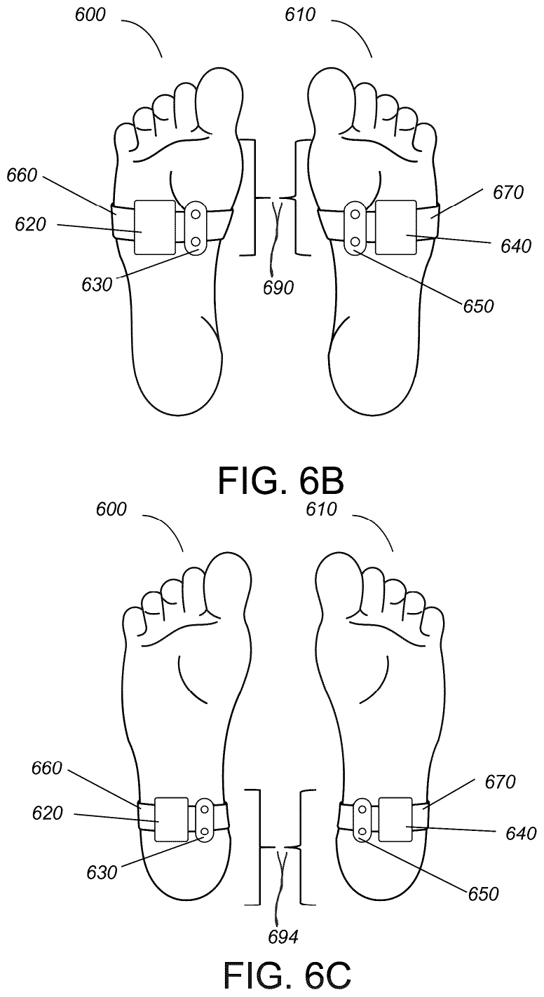

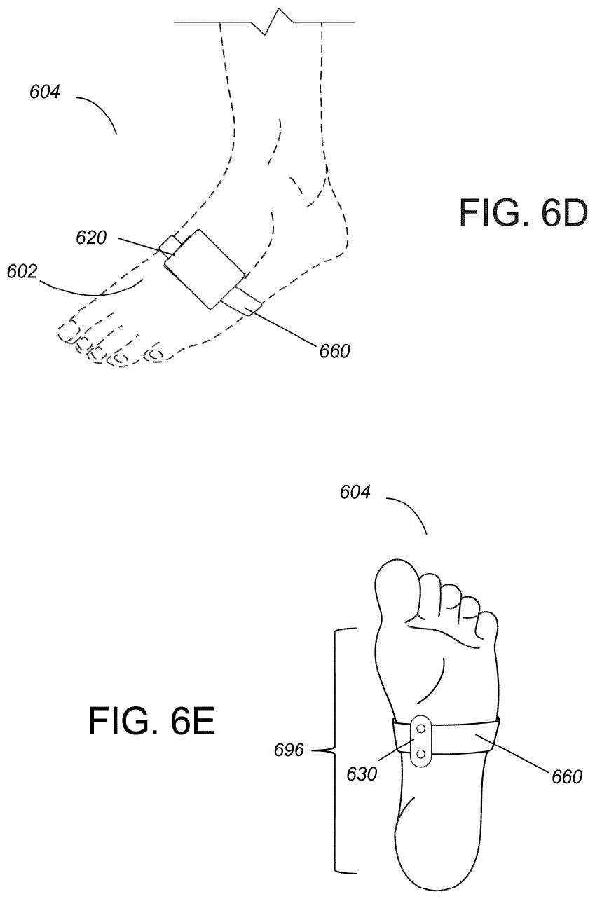

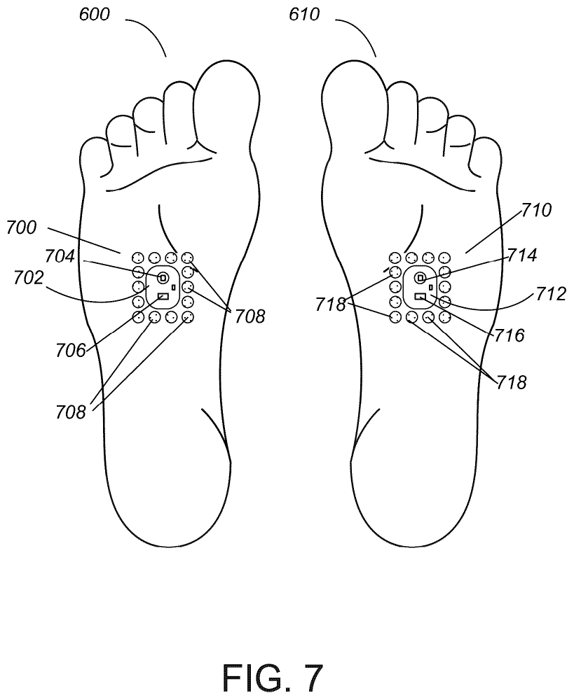

For example and as shown in FIGS. 6A-14, the wearable device 102 may be configured to be positioned on a sole of a foot, and the one or more electrodes or sensors are configured to be positioned on a dorsal region of the foot. In some implementations, wearable device 102 is configured to be positioned on a dorsal region of the foot, and one or more electrodes or sensors are configured to be positioned on a sole of the foot. In some implementations, wearable device 102 is configured to be positioned on a dorsal region of the foot, and one or more electrodes or sensors are configured to be positioned on the dorsal region of the foot. In some implementations, wearable device 102 is configured to be positioned on a sole of a foot, and one or more electrodes or sensors are configured to be positioned on the sole of the foot.

### Example Wearable Device I

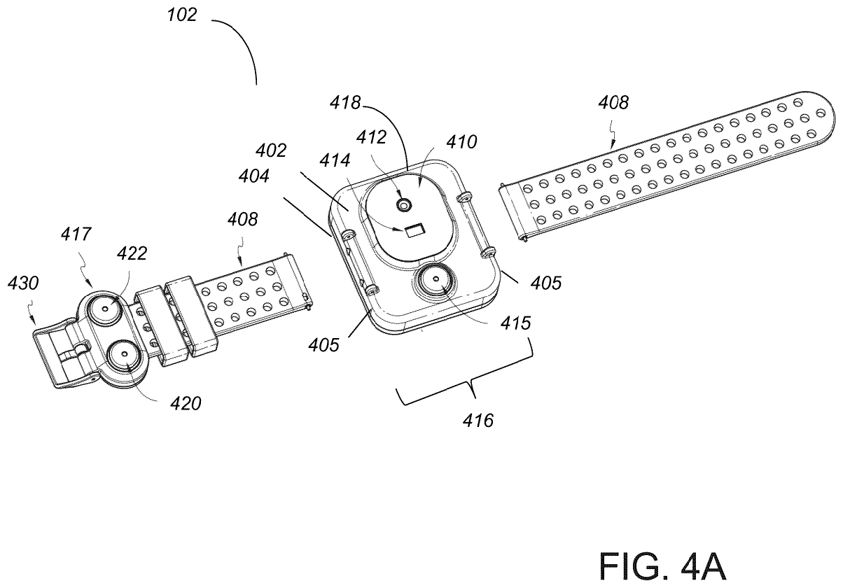

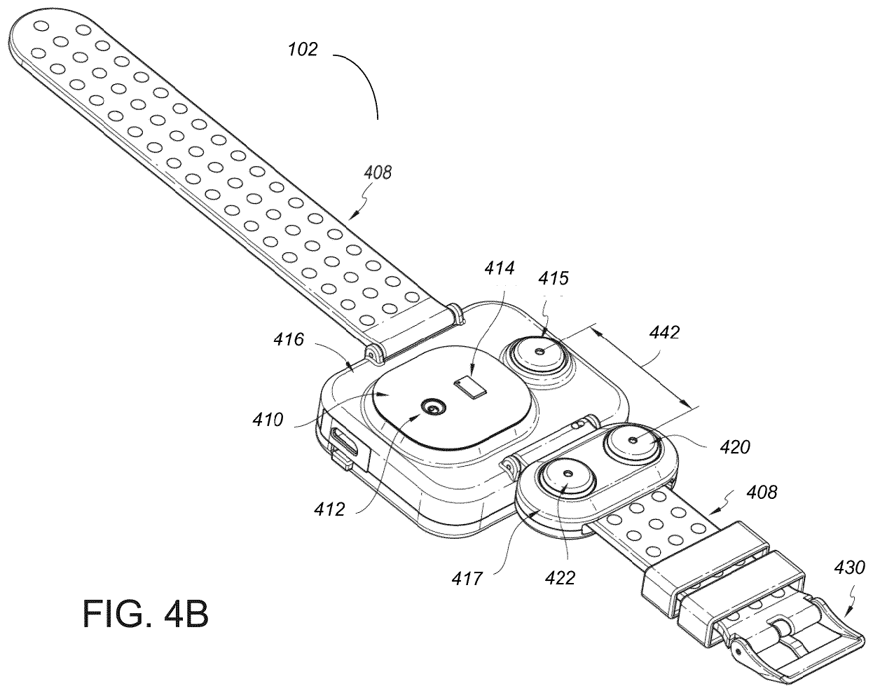

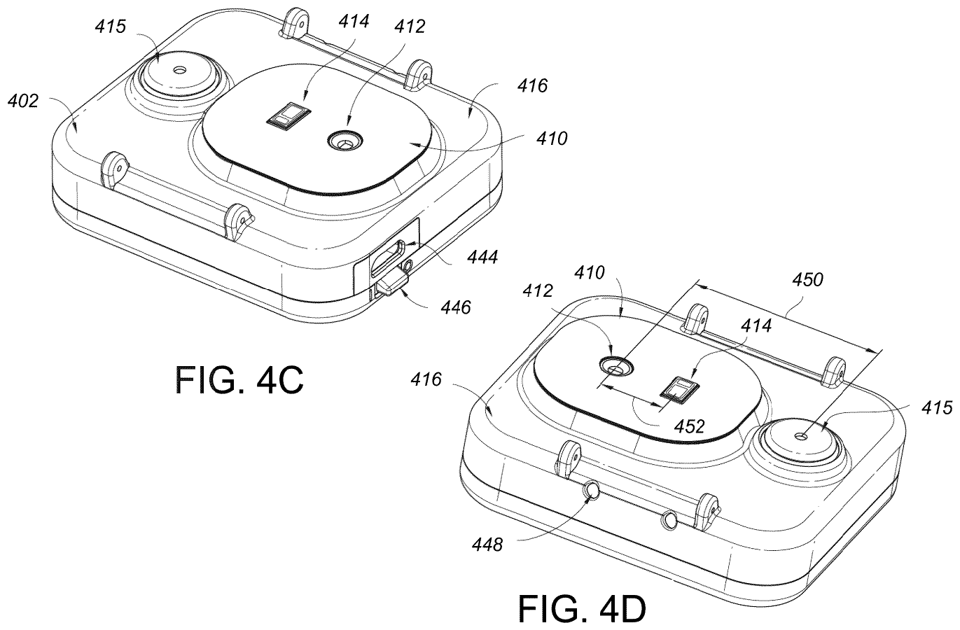

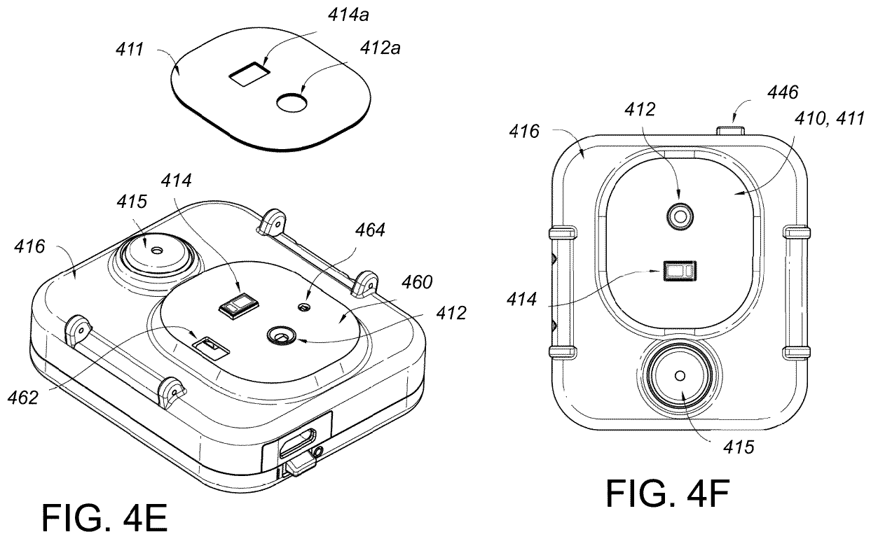

FIGS. 4A-4F illustrate a skin-facing side of an example device 102 configured to face a surface of a body (e.g., limb 302 or limb 304 of FIG. 3). As shown in FIG. 4A, the device 102 includes a body portion 416 having a first surface 404 opposite a second surface 402 configured to be in contact with a skin surface of a person. The first surface 404 and second surface 402 may be coupled via one or more or a plurality of sidewalls 405. For example, one or more sidewalls 405 may extend from a perimeter of the first surface 404 and couple to a perimeter of the second surface 402. The first surface 404 and/or second surface 402 may include one or more sensors positioned thereon. For example, one or more sensors on the first surface 404 may measure an environment of the person wearing or using the wearable device 102, and one or more sensors on the second surface 402 may measure one or more properties, features, or characteristics of the skin surface of the person. Alternatively, the first surface 404 may include one or more sensors or imagers or cameras for assessing a facial region of a person.

The wearable device 102 may include a stimulus source 410 (e.g., stimulus source 132 of FIG. 1B) in communication with the skin surface. In some implementations, a stimulus source 410 is positioned on a second surface 402 of the body portion 416, so that there is coupling or contact between the stimulus source 410 and a skin surface. Alternatively or additionally, a stimulus source 410 or one or more sensors (e.g., sensors 412, 414) and/or electrodes (e.g., electrode 415, 420, 422) may be positioned on a band 408 of the device 102, such that the body portion 416 is separate from a sensor module 418 (e.g., including one or more sensors 104, 412, 414, etc.) that includes the stimulus source 410 and the one or more sensors selected from sensors 104, for example. In some implementations, the stimulus source and/or one or more sensors may be distributed between the band, body, and sensor module depending on which sensors are incorporated into the system and their specific requirements or parameters. In some embodiments, the second surface 402 can include a recess or a cutout portion. The cutout portion can be positioned in different locations throughout the second surface. For example, the cutout can be at or around the center of the second surface. The cutout portion can comprise an inner perimeter. The cutout portion can be enclosed partially or entirely within an outer perimeter. The cutout portion can be configured to house one or more sensors.

The band 408 may be an adjustable or tensionable band including a buckle 430 on a first side and a series of receiving notches on a second, opposing side. Band 408 may allow for similar or alternative number and arrangements of electrodes 415, 420, and/or 422. In some implementations, a Velcro® or another hook-and-loop fastener (not shown), and/or a stretchable material (e.g., silicone, rubber, Lycra, Spandex, Elastane, neoprene, leather, fabric, etc.) can be employed along at least a portion of the band 408. The band may be configured to wrap around or coupled to a person at proximity of the person's foot (e.g., at or around shin of the person, an ankle of the person, a foot of the person, below the kneecap of the person or the like).

In some implementations, the band 408 may include connectors (not shown) for connecting electrodes (for example, electrodes 420 and 422) to other elements housed in the body 416 including, but not limited to, a power supply (for example, a battery) and a processor (for example, processor(s) 114 of FIG. 2). The connectors can be electrical traces, for example wires, conductive ink, circuitry or another connector, optical connectors, for example fiber optic cable, or other suitable connector that may be on, or at least partially embedded in a band 408. In some implementations, these traces from the electrodes 415, 420, and/or 422 of the band 408 to elements of the body 416 may be formed by cold molding or insert molding. In some implementations, connector wires may be threaded, woven, or sewn into the material of the band 408 and/or holes, channels, or other apertures in the band 408. In some implementations, the band 408 is at least partially made of a conductive material. In some implementations, connectors from a band 408 are connected to components (e.g., a battery, processor(s) 114) inside the body 416 through one or more holes (e.g., holes 448 of FIG. 4D) in the body 416.

In some embodiments, the band 408 may be integrated into a clothing item, such as a shoe, a tongue of a shoe, laces of a shoe, sides of a shoe such that the device 102 is positioned on a dorsal region of a foot, for example.

The band 408 may carry an electrode housing 417 that may hold additional electrodes, such as two electrodes 420 and 422. The electrode housing 417 may be integral with or formed as a unitary structure with the buckle 430. In some implementations, the electrode housing 417 may be openable and/or removable from the band 408. The electrode housing 417 may include a lower portion and upper portion for housing one or more electrodes, for example electrodes 420 and 422. The lower portion and/or upper portion may include features for securely seating and retaining electrodes and/or an end of band 408 and/or a buckle 430 and/or an upper portion of the band. These securing features can include recesses, notches, alignment pins, fasteners, and/or clips to hold and/or align the assembly components. In some implementations, the electrode housing 417 is secured to a particular location on the band 408, where the electrode housing 417 cannot slide (for example, with pins and holes 448). In some implementations, the lower portion and upper portion securely mate together and cannot be opened. In some implementations, the lower portion and upper portion securely mate together and can be opened by manipulating the pieces, for example, by sliding or twisting the pieces against each other, and/or opening a latch, button, or other retention feature. In some implementations, the electrode housing 417 is secured to the band 408 and can slide along the length of the band. For example, the lower portion and upper portion may secure together over and/or around the band 408 and provide a clamping force to the band 408 to secure the electrode housing 417 in position while being slidable along the band 408.

In some embodiments, electrode housing 417 is adjustable or positionable along a length of the band 408 such that the electrode housing is positioned on a different region of a body portion than body portion 416. For example, the electrode housing may be positionable on a dorsal region of a foot while the body portion is positionable on a sole of a foot. Further for example, the electrode housing may be positionable on a sole of a foot and the body portion may be positionable on a dorsal region of a foot (see, e.g., FIGS. 6D-6E). Still further for example, the electrode housing and the body portion may be on similar regions of the foot but offset from one another so that a stimulus source does not impact an electrode signal recording.

Referring again to FIG. 4A, the body portion 416 further includes a blood volume sensor 412 and, optionally, a skin temperature sensor 414. The blood volume sensor 412 can be integrated into a form factor such as the device 400 that improves continuous anomalous cardiac event monitoring. The blood volume sensor 412 can measure vasodilation response parameters. The skin temperature sensor 414 can also be integrated into the device 102. The skin temperature sensor 414 is positioned on the second surface 402 and can measure a temperature of the skin surface in contact with the stimulus source 410. The blood volume sensor 412 is positioned on the second surface 402 and can measure a blood volume of the skin surface. The blood volume sensor may be a photoplethysmography sensor or an impedance plethysmographic sensor. The blood volume sensor may employ light at one or more of 450-500 nm (blue), 500-570 nm (green), 570-610 nm (yellow), 610-760 nm (red), or infra-red (>760 nm) wavelength, or a combination thereof. Different wavelengths may be more appropriate for different applications, for example green (500-570 nm) light may be more accurate for heart rate measurements (e.g., heart rate variability, heart rate, etc.). In addition to, or alternatively, the blood volume sensor may also measure one or more of: heart rate, heart rate variability, or oxygen saturation.

FIG. 4B illustrates a perspective view of the wearable device of FIG. 4A. A second surface 402 of a body 416 is depicted. The second surface 402 of the body 416 may be similar or identical to a second surface 402 of other devices described herein, for example second surface 402 may include a heat source 410, blood volume sensor 412, and skin temperature sensor 414. In some implementations, the second surface 402 includes one electrode 415. A band 408 with a buckle 430 may include an electrode housing 417 that may carry additional electrodes, such as two electrodes 420 and 422. The electrodes 420 and 422 (and/or other additional electrodes and/or sensors) may be positioned on the second surface 402 and/or a tensionable band 408 of the device 102. The electrodes 415, 420, and/or 422 may be located away from the surface of the device 102. For example, electrode 415 may be placed on a raised platform 460 of the second surface 402 of the body 416. As discussed above with regard to electrodermal sensors 412 and 414, electrodes 415, 420, and/or 422 may be spaced at preselected distances. In some implementations electrodes 420 and 422 are spaced at a distance of about 5 mm to about 100 mm, for example 5 mm to about 10 mm, about 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, measured from a center point of each electrode 420, 422.

In some implementations, electrodes 420 and 422 may be spaced apart from the stimulus source 410 and/or electrode 415 by a distance 442, which can be about 10 mm to about 300 mm, for example about 10 mm to about 20 mm, about 20 mm to about 30 mm, about 30 mm to about 40 mm, about 40 mm to about 50 mm, about 50 mm to about 60 mm, about 60 mm to about 80 mm, about 80 mm to about 100 mm, about 100 mm to about 120 mm, about 120 mm to about 150 mm, about 150 mm to about 175 mm, about 175 mm to about 200 mm, about 200 to about 225 mm, about 225 mm to about 250 mm, about 250 mm to about 275 mm, about 275 mm to about 300 mm, measured from a center point of the electrodes 420, 422 to a center point of the heat source 410 and/or the electrode 415. Still further, electrode 415 may be spaced apart from a heat source 410 by a distance which can be about 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, measured from a center point of the electrode 415 and a center point of the heat source 410. When the body 416 is secured to the foot or ankle or a lower extremity with the band 408, in some implementations, the electrodes 420 and 422 are positioned on the band such that they are positioned at a region offset from the body portion 416.

FIG. 4C illustrates a partial view of the wearable device 102 of FIG. 4A. As shown, the body portion 416 of the device 102 includes a port 444 for electrically coupling the device 102 to a power source, for example to charge a battery (such as battery power 133) in the device 102. Additionally or alternatively, port 444 may electrically couple the wearable device to an external or remote computing device (e.g., laptop, desktop, server, workstation, etc.) to download data from the device or upload system parameters or install updates to the wearable device 102. Port 444 may also be used to connect auxiliary sensors, an input/output device (keyboard, joystick, buttons, switches, printer, camera, display), and/or memory unit. The wearable device 102 may further include one or more user interface elements 446, for example one or more buttons and/or switches, that may be used, for example, to power on and off the device, to input user specific reactions, features, or characteristics, to customize an interface or functionality of the user device, to mark events, to initiate pairing or data transfer, to call for help, etc. User interface elements 446 may alternatively or additionally include output and/or feedback elements, such as a speaker, light, and/or haptic stimulator. In some implementations, user interface element 446 may be used, for example, to indicate power on, charging, low battery, pairing mode, heating phase, malfunction, health event, and/or other status of the device 400 and/or user. In some implementations, user interface element 446 may be a feedback element that includes one or more LED behind a smoked, translucent, or transparent window. In some implementations, there is no display screen on the body 416.

FIG. 4D illustrates a rotated view of the wearable device 102 of FIG. 4C. As shown, the second surface 402 of body 416 may include a stimulus source 410, blood volume sensor 412, skin temperature sensor 414, and electrode 415, as discussed above. The second surface 402 of body 416 may be arranged to contact the dorsal or top side of the foot when worn to locate the stimulus source 410, blood volume sensor 412, skin temperature sensor 414, and electrode 415 generally along a midline of the top or dorsal side of the foot. Alternatively, the second surface 402 of body 416 may be arranged to contact the sole or bottom side of the foot when worn to locate the stimulus source 410, blood volume sensor 412, skin temperature sensor 414, and electrode 415 generally along a midline of the sole or bottom side of the foot. Blood volume sensor 412 and skin temperature sensor 414 may be placed within the stimulus source 410 spaced at a distance 452 from each other. Distance 452 may be about 10 mm to about 100 mm, for example 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, measured from a center point of the blood volume sensor 412 and a center point of the skin temperature sensor 414. Similarly, blood volume sensor 412 may be placed at a distance 450 from the electrode 415. Distance 450 may be about 10 mm to about 200 mm, for example 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, about 100 mm to about 120 mm, about 120 mm to about 140 mm, about 140 mm to about 160 mm, about 160 mm to about 180 mm, about 180 mm to about 200 mm, measured from a center point of the blood volume sensor 412 and a center point of the electrode 415.

FIG. 4E illustrates a perspective view and an exploded view of the wearable device 102 of FIG. 4C. As shown, the second surface 402 of the body 416 may include a raised platform 460. Platform 460 may include the stimulus source 410, blood volume sensor 412, and skin temperature sensor 414. In some implementations, the platform 460 may include electrode 415. Platform 460 may improve contact between the skin and the heat source 410, blood volume sensor 412, and skin temperature sensor 414. In some implementations, the platform 460 is sized to cover or substantially cover a portion of a dorsal region of a foot, a portion of a sole of a foot, or a portion of an ankle when device 102 is worn. In some implementations, the platform 460 is flexible and/or shaped, for example, curved, and may increase a contact area between the platform 460 and the skin when the device 102 is worn. In some implementations, stimulus source 410 symmetrically surrounds the blood volume sensor 412 and/or symmetrically surrounds the skin temperature sensor 414. In some implementations, stimulus source 410 may surround the blood volume sensor 412 with an area approximately equal to the area of stimulus source 410 that surrounds the skin temperature sensor 414.

In some implementations, the stimulus source 410 may include a warming plate 411, as shown in FIG. 4E. For example, the stimulus source 410 may include a warming plate 411 for increased heat distribution and/or heat retention. The second surface 402 of body 416 may include an opening, such as opening 462, to allow the stimulus source 410 and/or warming plate 411 to communicate with and/or access other components inside the body 416, for example the processor(s) 114 and battery power 133. The second surface 402 of the body 416 may include a thermistor 464 or other temperature sensor for monitoring the temperature of the stimulus source 410 and/or warming plate 411. The thermistor 464 may provide a heater temperature measurement, which may be used to control the stimulus source 410.

Warming plate 411 may include apertures for components surrounded and/or enclosed by the heat source 410, for example aperture 412a for blood volume sensor 412 and aperture 414a for skin temperature sensor 414. Aperture 412a and aperture 414a may allow improved contact between the skin and blood volume sensor 412 and skin temperature sensor 414. In some implementations, the blood volume sensor 412 includes two separate components, for example an emitter and a detector, and accordingly aperture 412a would include an aperture for each component. In general, heat source 410 may be a layered or laminate structure. In some implementations, the heat source 410 may include dimples and/or perforations for heat distribution and/or dissipation. Perforations may also improve adhesion.

FIG. 4F illustrates a front view of the wearable device 102 of FIG. 4C. The warming plate 411 is shown installed on heat source 410 of wearable device 102. Alternative or additional features, such as ridges, channels, fins, and the like may also be included on a heat source 410 to improve uniform heating and cooling and/or direct heating and cooling. For example, surface features may be used to direct heat from the heat source 410 toward or away from a lower extremity facing side, an ankle facing side, a foot facing side, or skin facing side of the device 102. The surface features may alternatively, or additionally, be located on a warming plate 411. Heat source 410 may be positioned on a raised platform 460 of the second surface 402 of the body 416. In some implementations, the heat source 410 is arranged farther from the hand than the electrode 415 when the device 102 is worn on the lower extremity.

### Example Wearable Device II

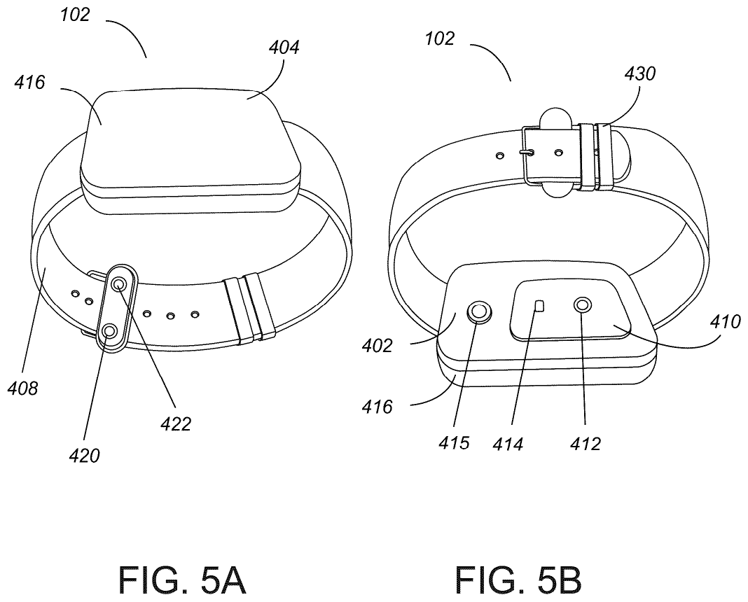

FIG. 5A illustrates a top perspective view of a wearable device 102 for measuring biological data on a lower extremity; and FIG. 5B illustrates a bottom perspective view of the wearable device 102 of FIG. 5A. The wearable device 102 shown in FIGS. 5A-5B may provide movable electrode and/or sensor portions. In addition, such devices may provide attachment features (e.g., bands, buckles, fittings, securing means, etc.) to enable a reliable and comfortable fit on the lower extremities. The comfort and reliability may ensure that the device 102 obtains proper signals from the skin surface site of the user wearing the device 102. For example, one or more attachment features may be provided on device 102 to enable a comfortable fit to allow for a variety of user differences in ankles, arches, heels, toes, or the like.

As shown in FIG. 5A, wearable device 102 includes a body portion 416 and a band 408 with a buckle 430 (or another securing means; any securing means is contemplated herein). A first side 404 of body portion 416 of device 102 faces away from a skin surface and may optionally have, indicators for various device functionality, or a display, for example. The band 408 may include one or more electrodes 420, 422. The electrodes may be movable along the length of the band to achieve proper positioning on the lower extremity. In some implementations, the electrodes 420, 422 may instead be located at a fixed location on the band to ensure consistent positioning relative to the body portion 416.

As shown in FIG. 5B, a second side 402 of body portion 416 of device 102 is configured to face a skin surface of the lower extremity for biological signal measurement. The second side 402 of body portion 416 includes a stimulus source 410 that at least partially surrounds blood volume sensor 412 and skin temperature sensor 414. The second side 402 may further optionally includes electrode 415, although variations without electrodes on the body portion 416 are also contemplated herein.

### Wearable Devices on Lower Extremities

FIGS. 6A-6E illustrate examples of wearable devices positioned on a lower extremity to measure biological data. FIG. 6A shows a right wearable device 620 positioned on a right lower extremity 600 and a left wearable device 640 positioned on a left lower extremity 610. Each wearable device 620, 640 may be secured to a respective lower extremity 600, 610, for example via a band 660, 670. One or more electrodes may also be positioned on each band. For example, a right securing means or band 660 may include one or more electrodes 630; and left securing means or band 670 may include one or more electrodes 650. As shown in this embodiment, each wearable device 620, 640 is configured to be in contact with a sole of a foot 600, 610, respectively, and more particularly, an arch region 680 of a foot 600, 610. In another embodiment, as shown in FIG. 6B, each wearable device 620, 640 is configured to be in contact with a metatarsal region 690 of a foot 600, 610. In still another embodiment, as shown in FIG. 6C, each wearable device 620, 640 is configured to be in contact with a heel region 694 of a foot 600, 610.

FIGS. 6D-6E show another embodiment in which the wearable device 620 and one or more electrodes 630 are positioned on opposite sides of a lower extremity. For example, wearable device 620 may be positioned on a top or dorsal region 602 of a foot 604 and one or more electrodes 630 may be positioned on a bottom side or sole region 696 of the same foot 604. In one embodiment, both the wearable device 620 on the dorsal region 602 and one or more electrodes 630 positioned on a sole 696 are secured to the foot via band 660. Alternatively, wearable device 620 may be secured to the foot via a first securing means or band and the one or more electrodes 630 may be secured to the foot via a second securing means or band. In a further embodiment, the wearable device may be secured to the sole of the foot and the one or more electrodes may be secured to the dorsal region.

### Example Patch Wearable Devices on Feet

FIG. 7 illustrates another embodiment of a first and second wearable device positioned on the feet to measure biological signals. The wearable device shown in FIG. 7 may be a patch or an adherable device, arranged to be secured to a lower extremity, for example a foot. In one embodiment, the device is worn unilaterally; in another embodiment, the device is worn bilaterally. As shown in FIG. 7, right wearable device 700 may be worn on a right lower extremity 600 and a left wearable device 710 may be worn on a left lower extremity 610. Right wearable device 700 includes a blood volume sensor 704, a stimulus source 702, and a skin temperature sensor 706, as described elsewhere herein. Wearable device 700, in some embodiments, may further optionally include a plurality of electrodes and/or sensors 708, such that a plurality of biological signals may be measured with one device. Each electrode or sensor of the plurality of electrodes or sensors 708 may be switched on or off in a preselected sequence, in response to a stimulus event from the stimulus source 702; in response to received biological signals; or the like. Similarly, left wearable device 710 includes a blood volume sensor 714, a stimulus source 712, and a skin temperature sensor 716, as described elsewhere herein. Wearable device 710, in some embodiments, may further optionally include a plurality of electrodes and/or sensors 718, such that a plurality of biological signals may be measured with one device. Each electrode or sensor of the plurality of electrodes or sensors 718 may be switched on or off in a preselected sequence; in response to a stimulus event from the stimulus source 702; in response to received biological signal, or the like.

### Example Patch Wearable Devices on Feet (Mid-Arch Region Placement)

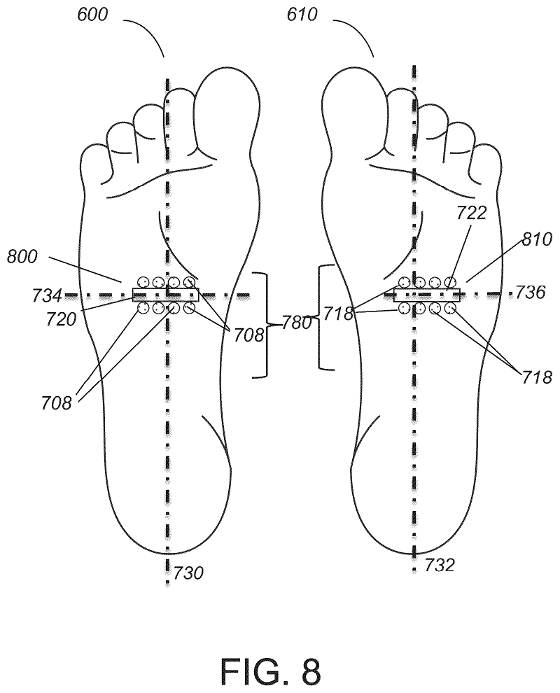

FIG. 8 illustrates yet another embodiment of a first and second wearable device positioned on a lower extremity to measure biological signals. Similar to FIG. 7 above, this embodiment includes patches or adherable devices arranged on each of a left and right foot. In this example, the devices are located in the middle arch region of each respective foot with the sensors arranged to obtain measurements from such regions. For example, a right device 800 is shown positioned on a right lower extremity 600, and a left device 810 is shown positioned on a left lower extremity 610. Right device 800 includes a body 720 and a plurality of electrodes or sensors 708 positioned on, about or integrated into body 720. The plurality of electrodes or sensors 708 can measure one or more biological signals of a patient, such as any of the signals described elsewhere herein. In one embodiment, the one or more biological signals may be used to determine a gait of the patient and/or to identify and remove motion artifacts from the various signals. Left device 810 includes a body 722 and a plurality of electrodes or sensors 718 positioned on, about, or integrated into body 722. The plurality of electrodes or sensors 718 can measure one or more biological signals of a patient, such as any of the signals described elsewhere herein.

Although the embodiment of FIG. 8 is shown adhered to the arch region 780, the present disclosure further encompasses similar embodiments that are positioned in a metatarsal region, heel region, sole, dorsal region, ankle, and the like. Further, as shown in FIG. 8, each wearable device 800, 810 is positioned such that a long axis 734, 736, respectively, of each device is substantially perpendicular to a parasagittal plane 730, 732 of each foot 600, 610, respectively.

### Example Patch Wearable Devices on Feet (Lower Arch Region Placement)

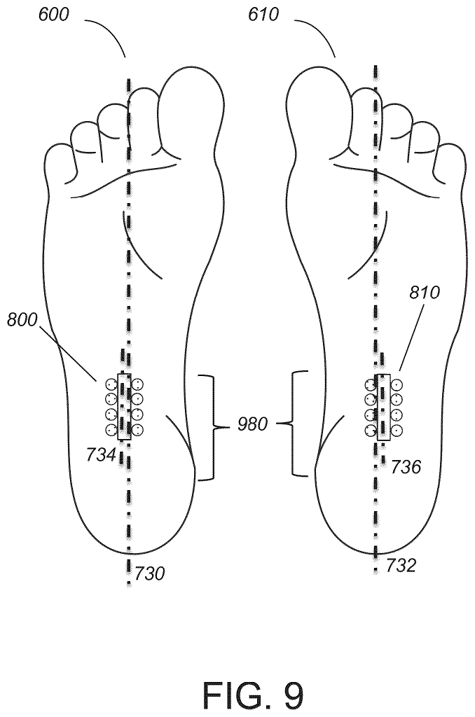

FIG. 9 illustrates the first and second wearable device 800, 810 of FIG. 8 positioned in a different orientation and location on a left foot 610 and a right foot 600, respectively. In this embodiment, the devices 800, 810 are located on the lower arch region of each respective foot with the sensors arranged to obtain measurements from such regions. As shown in FIG. 9, a long axis 734, 736 of each device 800, 810, respectively, is substantially parallel to a parasagittal plane 730, 732 of each foot, respectively. Further, each device 800, 810 is positioned in a lower arch region or an upper heel region 980.

### Example Wearable Devices on Ankle

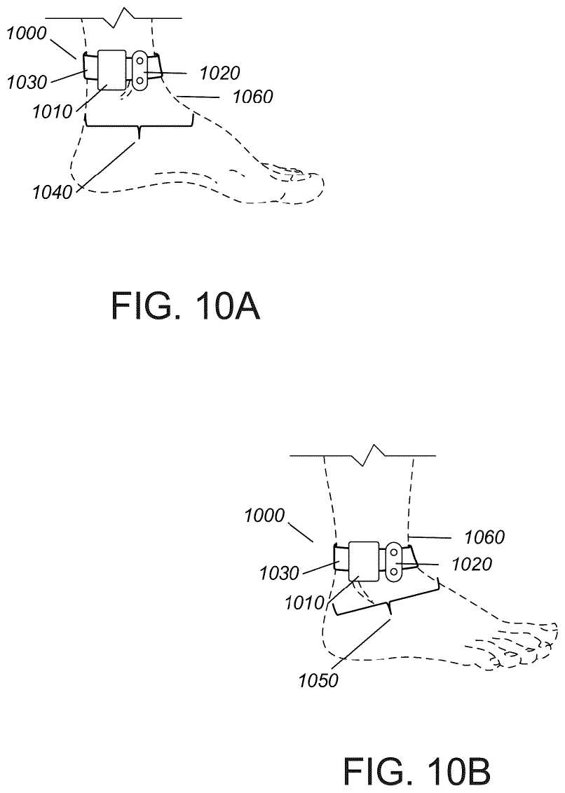

FIGS. 10A-10B illustrate embodiments of a wearable device positioned on an inner portion and an outer portion of an ankle, respectively. As shown in FIGS. 10A-10B, wearable device 1000, comprising a body portion 1010 and one or more electrodes 1020, may be secured, via securing means 1030, to an inner portion 1040 of an ankle 1060 (FIG. 10A) or an outer portion 1050 of an ankle 1060 (FIG. 10B). In another embodiment, the body portion may be positioned on an inner portion 1040 of an ankle and the one or more electrodes may be positioned on an outer portion 1050 of the ankle. In still another embodiment, the body portion may be positioned on an outer portion 1050 of an ankle and the one or more electrodes may be positioned on an inner 1040 portion of the ankle. In additional embodiments, one or both of the wearable body portion 1010 and the one or more electrodes 1020 may be positioned on a front portion or region of an ankle.

### Example Wearable Devices in Shoe

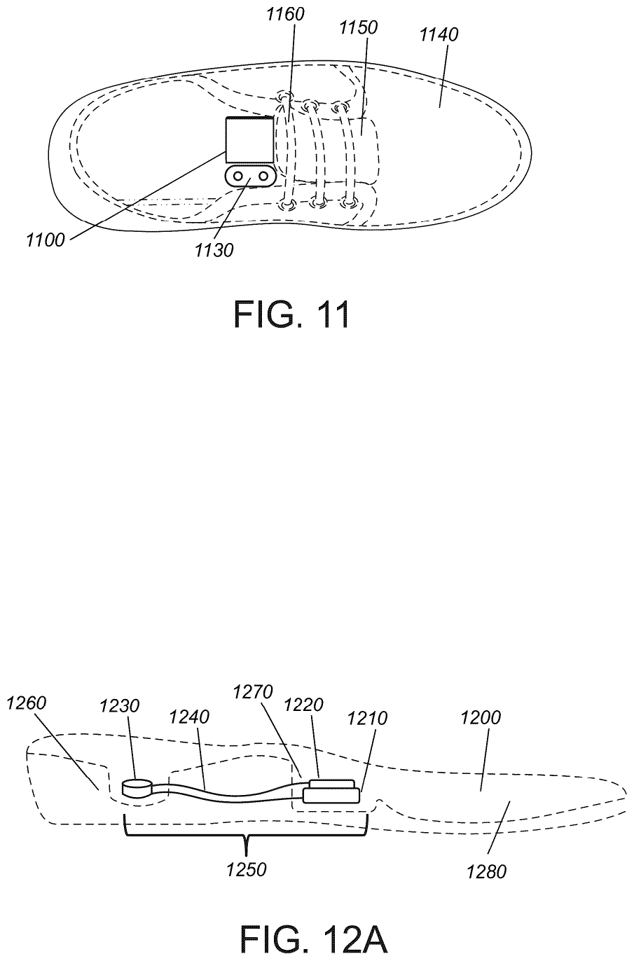

FIG. 11 illustrates another embodiment of a wearable device positioned on the top of the feet adjacent to a shoe top. The shoe includes laces 1160 and tongue 1150, which each contact a top or a dorsal region of a foot when the foot is positioned in the shoe. In one embodiment, a wearable device is integrated into a shoe 1140 such that the wearable device 1100 is positioned to be in contact with a skin surface of a top or dorsal region of the foot positioned in the shoe. Further, one or more electrodes 1130 may further be positioned in shoe 1140 and in contact with a skin surface of the top or dorsal region of the foot positioned in the shoe 1140. The wearable device 1100 may be integrated into the laces 1160, tongue 1150, or another structure of the shoe 1140 such that the wearable device is positioned against a top or dorsal region of a foot positioned in the shoe 1140.

### Example Wearable Devices in Shoe Insert

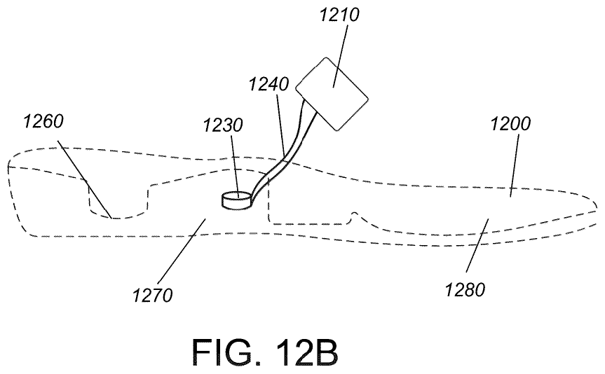

FIGS. 12A-12B illustrate another embodiment of a wearable device 1250 integrated in a shoe insert 1200. Wearable device 1250 includes body portion 1210 comprising one or more electrodes or sensors 1220 that is electrically coupled, via wires 1240, to one or more additional sensors or electrodes 1230. As shown in FIG. 12A, the one or more additional sensors or electrodes 1230 may be at least partially embedded in a heel region 1260 of insole or insert 1200 and a body portion of device 1250 may be at least partially embedded in an arch region 1270 of insole 1200. Alternatively, the one or more additional electrodes or sensors may also be positioned in an arch region 1270 or a metatarsal region 1280 of the insole 1200 and/or the body portion 1210 may be positioned in a heel region 1260 or metatarsal region 1280 of insole 1200. The body portion 1210 and one or more additional electrodes or sensors 1230 may be co-located in insole 1200 or located in separate regions of insole 1200. The configuration of the wearable device 1250 in FIG. 12B is similar to that of FIG. 12A except that only a portion of the device is at least partially embedded in or integrated into insole 1200. As shown in FIG. 12B, one or more additional sensors or electrodes 1230 are at least partially integrated into or embedded into insole 1200 while one or more electrical connections 1240 extend from the insole 1200 and connect to body portion 1210 of wearable device. The electrical connections 1240 may be embedded in or wrapped in a fabric or material or a shoe in which the insole 1200 is positioned. In such a configuration as shown in FIG. 12B, body portion 1210 may be in contact with a dorsal region of a foot while one or more sensors or electrodes 1230 are in contact with an arch region 1270 of the foot. Alternatively, the one or more additional electrodes or sensors 1230 may be at least partially embedded or integrated into a heel region 1260 or a metatarsal region 1280 of insole 1200.

The insole or insert 1200 may include silicone, elastane, or a combination thereof. In some embodiments, the shoe insert is porous. Additionally, or alternatively, the insole or insert may be at least partially gel filled or include highly compressive foam to maintain pressure contact between the sensors and the skin surface of the lower extremity throughout a range of foot sizes and shapes. Additionally, or alternatively, the insole or insert or a shoe may include or define one or more cavities such that various components can be removably positioned in the cavities, such that the insole or insert can be replaced, switched, washed, etc. In some embodiments, the sensors and/or electrodes can be decoupled from the housing (e.g., body portion) and wired to the electronics (e.g., processor, memory, power source, etc.) to debulk the system and physically distribute components within the insole or insert. In still additional embodiments, a power source may be positioned apart from the sensors, electrodes, stimulus source, etc. since the battery may need to be replaced or recharged and/or since the battery may be larger in size than the rest of the components so it could be positioned in a less sensitive skin location. In some implementations, recharging the device 125 may be performed wirelessly, for convenience.

In some implementations, the device may be switched on at the power source to begin obtaining measurements and may be switched off at the power source to stop obtaining measurements. In some implementations, the switching on and/or off may be performed without the use of a physical switch. For example, the device 1250 may be activated to begin obtaining measurements based on algorithmic sensing of a foot being present in a shoe associated with the device 1250. In another example, the detection may function with a confirmation signal to a companion device (e.g., a mobile device, or the like). In some implementations, the detection may function without a confirmation signal to the companion device.

Optionally, an insert or insole may be specifically made and/or sized for a particular patient. For example, a lower extremity, limb, etc. may be scanned by a three-dimensional scanner such that the insole is tailored for the shape and size of the limb, lower extremity, etc. and/or the sensor or electrode placement is specifically tailored for the limb or lower extremity or even how a patient walks or stands.

### Example Wearable Devices on Top of Foot

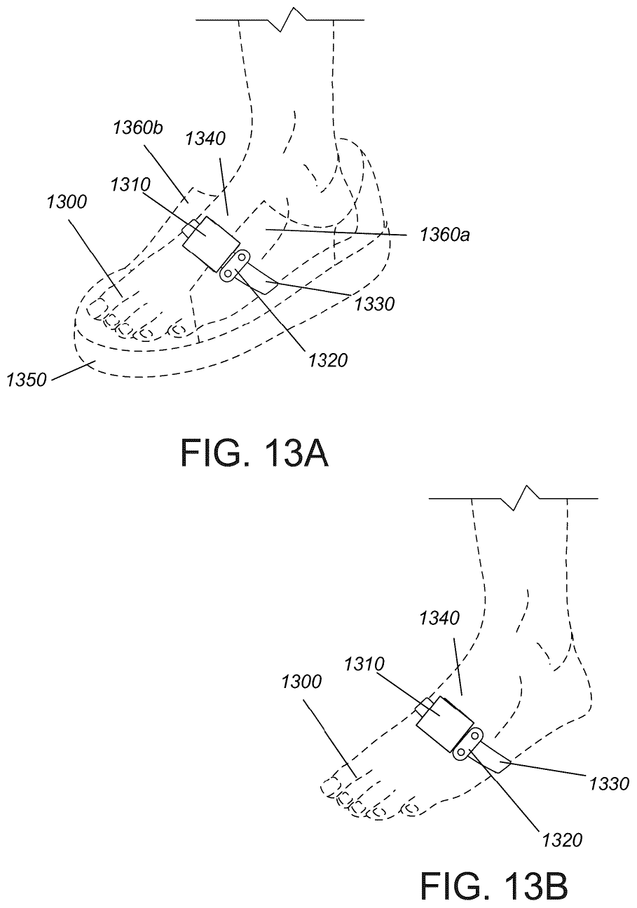

FIGS. 13A-13B illustrate the devices of FIGS. 5A-5B strapped to a top portion of a foot in two different configurations. FIGS. 13A-13B illustrate a body portion 1310 of a wearable device, as described elsewhere herein, that is configured to be positioned on a top or dorsal region 1340 of a foot 1300. In the embodiment of FIG. 13A, the band 1330 that is coupled to the body portion 1310 and one or more electrodes or sensors 1320 is integrated into one or more flaps 1360 or portions of a shoe 1350, sandal, slipper, or the like. For example, in the embodiment of FIG. 13A, the band 1330 may be configured as a strap to maintain the foot 1300 in the shoe 1350 during ambulation or when reposed. The band 1330 may extend from a first flap 1360a and irreversibly or reversibly couple to a second flap 1360b. The band 1330 may include a stretchable material, may include one or more attachment mechanisms (e.g., buttons, hook and loops, fasteners, buckles, etc.), or the like. In the embodiment of FIG. 13B, the band 1330 may circumscribe the foot 1300 to couple the body portion 1310 and one or more electrodes or sensors 1320 to the foot 1300. In such a configuration, the body portion 1310 of the wearable device and the one or more electrodes or sensors 1320 may be coupled to a dorsal or top region of foot 1340.

### Example Devices on Scale

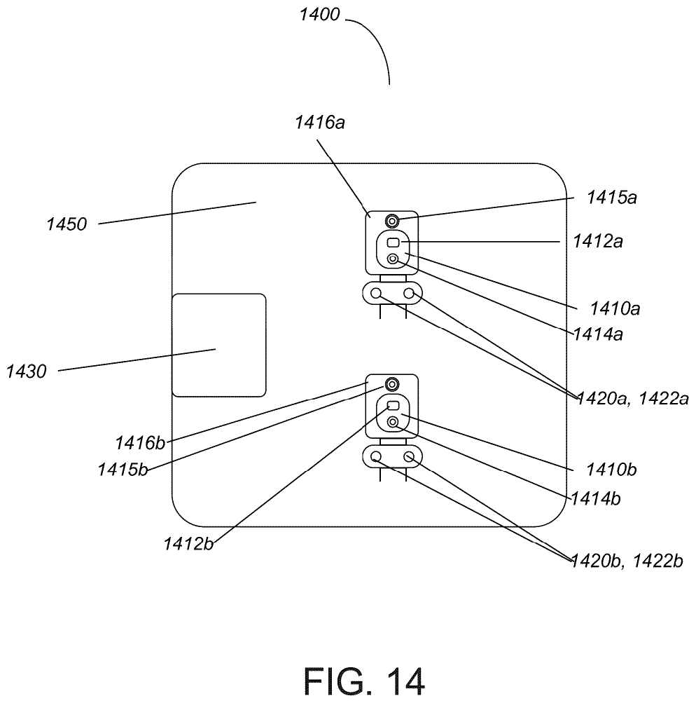

FIG. 14 illustrates another embodiment of a wearable device configured as part of a platform 1400 configured for a user or patient to stand on for biological signal measurement. A surface material 1450 of platform 1400 may be transparent or translucent such that the measurement devices may be visible therethrough to the user so that the user can accurately position his or her limbs, hands, feet, etc. on the platform for measurement or assessment. In other embodiments, the material may be opaque such that the surface material 1450 includes visual indicators for limb, hand, foot, etc. placement for optimal biological signal measurement or assessment. Each measurement device may be integrated into or embedded in the platform for biological signal measurement or assessment. Each measurement device, for a right area and left area, includes a body portion 1416a, 1416b comprising a stimulus source 1410a, 1410b, a blood volume sensor 1412a, 1412b, a skin temperature sensor 1414a, 1414b, and optionally an electrode 1415a, 1415b, respectively. In some embodiments, the one or more additional sensors or electrodes 1420a, 1420b, 1422a, 1422b are also embedded in the platform or integrated into the platform for additional biological signal measurement. The platform may optionally further include a display 1430 for displaying a weight measurement (in embodiments where the platform further functions as a scale) or one or more biological parameters that have been measured by the one or more measurement devices integrated into the platform 1400.

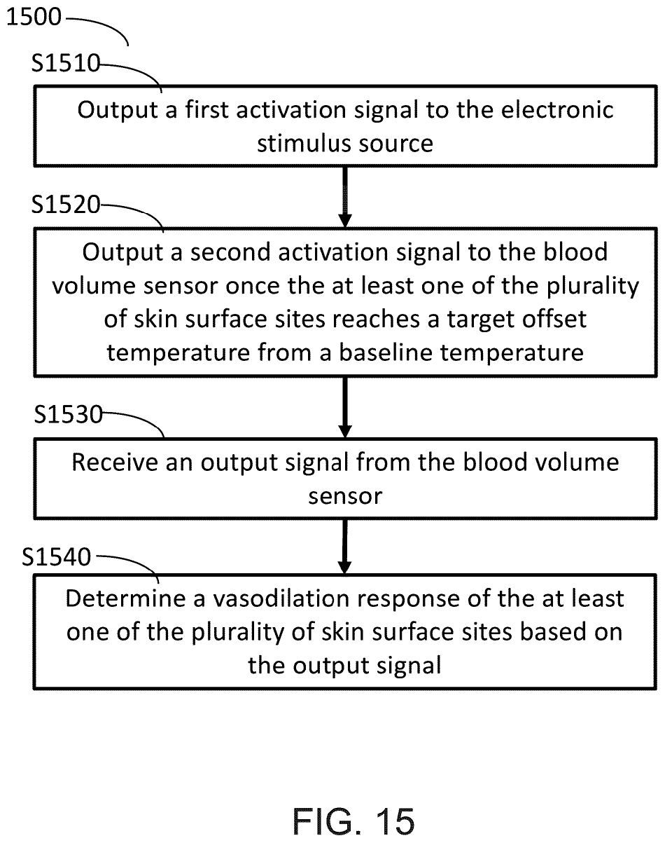

FIG. 15 illustrates a flow diagram of an example process 1500 for characterizing or monitoring a vasodilation response of a person. In some embodiments, the process 1500 may be performed by a wearable device positioned on or adjacent to a lower extremity of a person to monitor a plurality of skin surface sites of the person. The wearable device may include a thermal stimulus source having a surface area to provide thermal stimulus, a blood volume sensor to sense a vasodilation response of at least one of the plurality of skin surface sites, and at least one processor communicatively coupled to the wearable device to: output a first activation signal to the thermal stimulus source at block S1510, output a second activation signal to the blood volume sensor once the at least one of the plurality of skin surface sites reaches a target offset temperature from a baseline temperature at block S1520, receive an output signal from the blood volume sensor at block S1530, and determine a vasodilation response of the at least one of the plurality of skin surface sites based on the output signal at block S1540. The process 1500 may further include outputting an activation signal to a skin temperature sensor to monitor a temperature of the at least one of the plurality of skin surface sites and receiving an input when the at least one of the plurality of skin surface sites reaches the target offset temperature.

The wearable device may optionally further include one or more sensors or electrodes that may record bioelectrical signals. One or more electrodes may be positioned to be in contact with at least one site of the plurality of skin surface sites. In such embodiments, the process 1500 performed by the processor may further include outputting an activation signal to the one or more sensors or electrodes to begin recording bioelectrical signals from at least one site of the plurality of skin surface sites and receiving an input of bioelectrical signals from the one or more sensors or electrodes. The processor may further be configured to process the signals and output an indication related to the measured signals. For example, the indication may be a respiration rate, a pulse rate, a heart rate variability, a stress indication, a motion of the patient, etc.

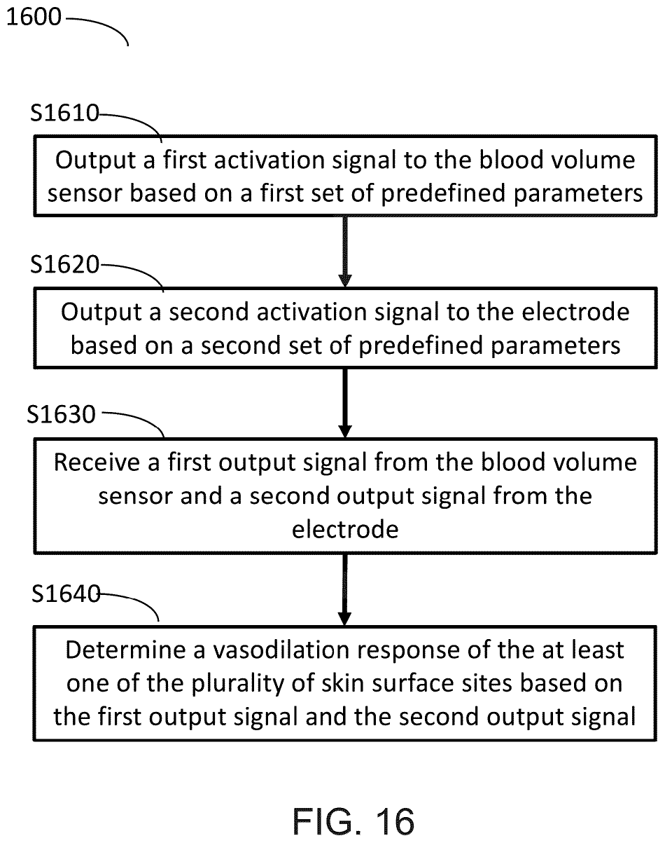

FIG. 16 illustrates a flow diagram of an example process 1600 for characterizing or monitoring a vasodilation response of a person. In some embodiments, the process 1600 may be performed by a wearable device positioned on or adjacent to a lower extremity of a person and to monitor a plurality of skin surface sites of the person. The wearable device may include a blood volume sensor to sense a vasodilation response of at least one of the plurality of skin surface sites, an electrode to record bioelectrical signals, the electrode being placed in contact with at least one site in the plurality of skin surface sites, and at least one processor communicatively coupled to the wearable device to: output a first activation signal to the blood volume sensor based on a first set of predefined parameters at block S1610, output a second activation signal to the electrode based on a second set of predefined parameters at block S1620, receive a first output signal from the blood volume sensor and a second output signal from the electrode at block S1630, and determine a vasodilation response of the at least one of the plurality of skin surface sites based on the first output signal and the second output signal at block S1640. The process 1600 may further include combining or comparing the first output signal and the second output signal to determine the vasodilation response. For example, in embodiments in which the electrode includes an electrodermal activity electrode (EDA), signals from the EDA electrode and the blood volume sensor may be compared and/or combined to determine a vasodilation response of at least one of the skin surface sites.

In some implementations, a first activation signal may include detection, by a sensor of device 102, of a weight or presence on or near the sensor. For example, a weight may be detected at a sensor of a scale device utilizing device 102. In another example, a skin surface may be detected against a surface of device 102. Upon detecting the skin surface against (adjacent, touching, etc.) a sensor of the device 102, for example for a predefined period of time (e.g., about 30 seconds to about 90 seconds), may trigger the device 102 to begin taking measurements of the skin surface.

The systems and methods of the implementations described herein and/or variations thereof can be embodied and/or implemented at least in part as a machine configured to receive a computer-readable medium storing computer-readable instruction. The instructions are executed by computer-executable components preferably integrated with the system and one or more portions of the hardware processor on the device for detecting stroke, neurological decline, and/or computing device. The computer-readable medium can be stored on any suitable computer-readable media (e.g., memory 118) such as RAMs, ROMs, flash memory, EEPROMs, optical devices (e.g., CD or DVD), hard drives, floppy drives, or any suitable device. The computer-executable component can be a general or application-specific hardware processor, but any suitable dedicated hardware or hardware/firmware combination can alternatively or additionally execute the instructions.

### Example Wearable Devices in Sock

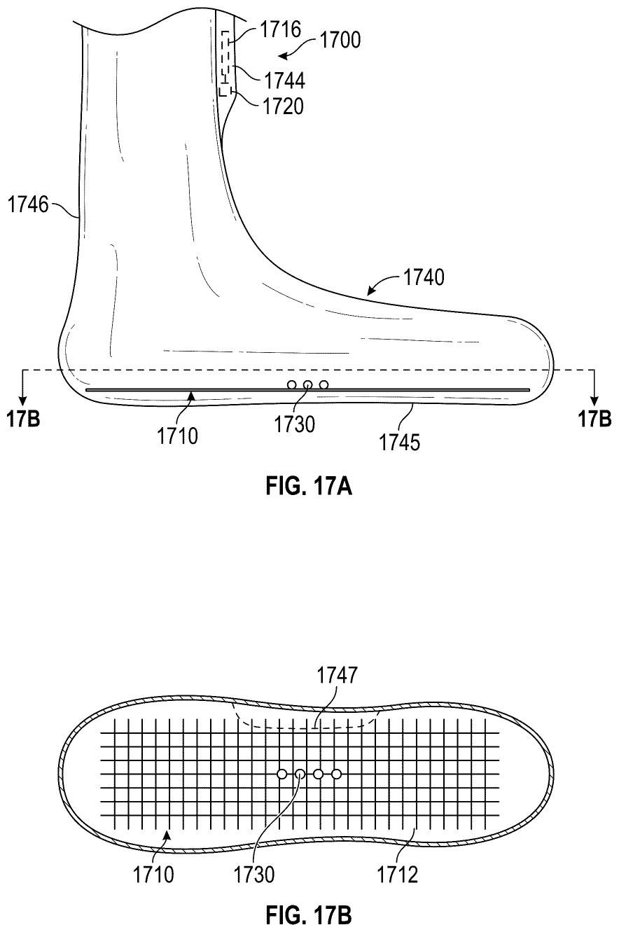

FIGS. 17A-17B illustrate an example device 1700 configured to be worn with a sock (or a pair of socks), such as sock 1740. In some embodiments, the device 1700 can have a first body 1716, a second body 1720, a sensor 1730, and a heat source 1710. The sensor 1730 can comprise one or more sensors positioned next to each other or spaced a part from each other. In some embodiments, the first body 1716 can have some (or all) features and components of the body portion 416, and the second body 1720 can have some (or all) features of the electrode housing 417 discussed with respect to FIG. 4A. The first body 1716 and the second body 1720 can communicate with each other and/or other components of the device 1700 (such as a sensor 1730, heat source 1710, etc.) mechanically or wirelessly. For example, the heat source 1710 can communicate with the first body 1716 and/or second body 1720 mechanically (e.g., via hard wire connection) or wirelessly (e.g., via Bluetooth). Similarly, the sensor 1730 can communicate with the first body 1716 and/or second body 1720 mechanically (e.g., via hard wire connection) or wirelessly (e.g., via Bluetooth).

Still referring to FIGS. 17-A-17B, the sock 1740 can be configured to be worn with a right foot and/or a left foot or both. The sock 1740 can have a bottom portion 1745, a top portion 1746, and a pocket 1744. The first body 1716 and the second body 1720 can be positioned in the pocket 1744. This can enable the user to have bulky components of the device 1700 in a pocket that may not be in continuous pressure as the user steps on a ground, which can be uncomfortable in use. On the other hand, the heat source 1710 can be positioned on (or integrated with) the bottom portion 1745 of the sock 1740. In some embodiments, the heat source 1710 can comprise a heater trace layer 1712. The heater trace layer 1712 may be interconnected with each other and/or integrated with the bottom portion 1745 of the sock 1740. The heater trace layer 1712 can cover the bottom portion 1745 in its entirety or in part. The heater trace layer 1712 can be spread over all (or part of) bottom portion 1745 to form a thin layer that does not make it uncomfortable for the user when the user wears the sock 1740. The heater trace layer 1712 may include a single serpentine trace structure (e.g., wires, circuitry, and the like) that spread over a part (or all) of the bottom portion 1745. The heater trace layer 1712 can be manufactured to have a variety of different patterns depending on a user's need. For example, they can have a checkered patterned with individual trace elements approximately perpendicular to each other. In some embodiments, the heat source 1710 is not limited to the bottom portion of the sock 1740 and can be configured with other parts of the sock 1740 (e.g., at the top portion 1746, around the toe area etc.). In some embodiments, the heater trace layer 1712 may have one or more openings to accommodate the sensor 1730. In some embodiments, the sensor 1730 can be positioned in an arch portion 1747 (corresponding to an arch recess on a sole of a foot) or can be manufactured such that they are within the serpentine trace structure positioned on the bottom portion 1745. In some embodiments, the sensor 1730 can be positioned symmetrical along a longitudinal or transverse axis of the foot. The heater trace layer 1712 and/or the sensor 1730 can be manufactured to be intertwined, sewn, glued, integrated etc. with the components (such as fabrics) of the sock 1740.

### Another Example Wearable Device in Shoe

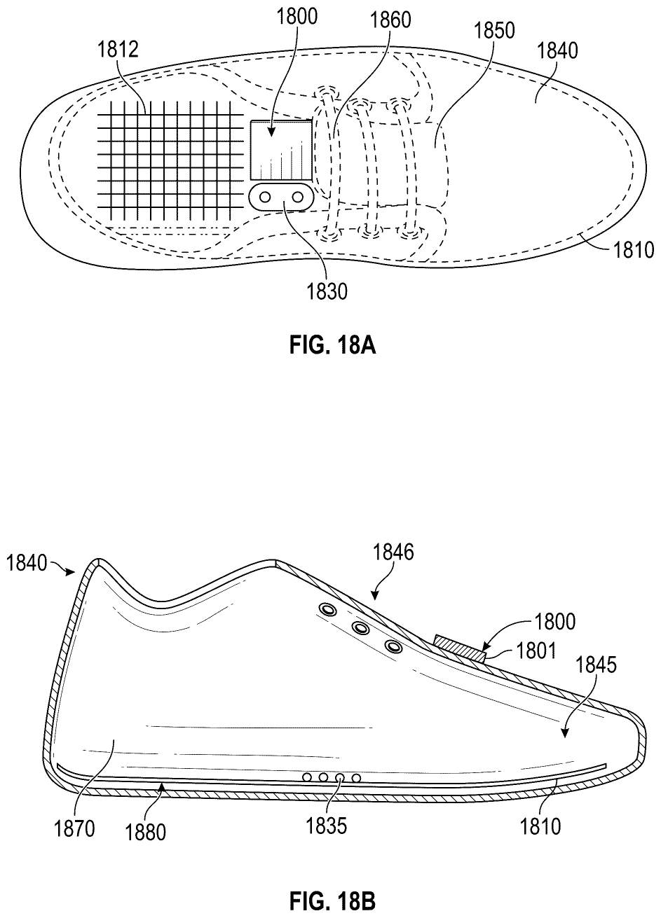

FIGS. 18A-18B illustrate an example wearable device 1800 configured to be worn with a shoe (or a pair of shoes), such as shoe 1840. The shoe 1840 can have tongue 1850, laces 1860, hill portion 1870, and a sole portion 1880. In some embodiments, a body portion 1801 and/or electrode 1830 can be positioned on a top portion 1846 of a shoe 1840 adjacent to the tongue 1850 and/or the laces 1860. A heater source 1810 having a heater trace layer 1812 can be positioned on a bottom portion of the shoe 1840. In some embodiments, the heater source can be a shoe insert and/or integrated with a shoe insert. The heater trace layer 1812 can cover the bottom portion 1845 in its entirety or in part. The bottom portion 1845 can be the bottom of the shoe 1646 or can be a shoe insert. The heater trace layer 1812 can be spread over all (or part of) bottom portion 1845 to form a thin layer that does not make it uncomfortable for the user to wear the shoe 1840. The heater trace layer 1812 may include a single serpentine trace structure (e.g., wires, circuitry, and the like) that spread over a part (or all) of the bottom portion 1845. The heater trace layer 1812 can be manufactured to have a variety of different patterns depending on a user's need. For example, they can have a checkered patterned with individual trace elements approximately perpendicular to each other. In some embodiments, the heater trace layer 1812 may have one or more openings to accommodate one or more sensor 1835. The sensor 1835 can be positioned (adjacent to each other spread from one another) at the bottom portion 1845. In some embodiments, the sensor 1835 (and/or body portion 1801 and/or electrode 1830) can be positioned in the hill portion 1870.

Sill referring to FIGS. 18A-18B, the shoe 1840 can be configured to be worn with a right foot and/or a left foot or both. An insert portion (not shown) such as an insole can be placed in the interior space of the shoe 1840 and cover (fully or partially) the heater source 1810. In some other embodiments, the heater source 1810 can be integrated (or intertwined with) with the insert portion. The body portion 1801 and the electrode 1830 can communicate with each other and/or other components of the wearable device 1800 (such as the sensor 1835 or the heater source 1810) mechanically or wirelessly. For example, the heater source 1810 can communicate with the body portion 1801 and/or the electrode 1830 mechanically (e.g., via hard wire connection) or wirelessly (e.g., via Bluetooth). Similarly, the sensor 1835 can communicate with the body portion 1801 and/or the electrode 1830 mechanically (e.g., via hard wire connection) or wirelessly (e.g., via Bluetooth).

As used in the description and claims, the singular form “a”, “an” and “the” include both singular and plural references unless the context clearly dictates otherwise. For example, the term “signal” may include, and is contemplated to include, a plurality of signals. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5 percent, 1 percent or 0.1 percent. All numerical ranges provided herein are inclusive of the stated start and end numbers. The term “substantially” indicates mostly (i.e., greater than 50%) or essentially all of a device, substance, or composition.

The term “horizontal” as used herein is defined as a plane parallel to the conventional plane or surface of a heating element (e.g., heat source 410), regardless of its orientation. The term “vertical” refers to a direction perpendicular to the horizontal as just defined. Terms, such as “on”, “above”, “below”, “bottom”, “top”, “side” (as in “sidewall”), “higher”, “lower”, “over”, and “under”, are defined with respect to the horizontal plane.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Implementations defined by each of these transitional terms are within the scope of this disclosure.

The examples and illustrations included herein show, by way of illustration and not of limitation, specific implementations in which the subject matter may be practiced. Other implementations may be utilized and derived therefrom, such that structural and logical substitutions and changes may be made without departing from the scope of this disclosure. Such implementations of the inventive subject matter may be referred to herein individually or collectively by the term “invention” merely for convenience and without intending to voluntarily limit the scope of this application to any single invention or inventive concept, if more than one is in fact disclosed. Thus, although specific implementations have been illustrated and described herein, any arrangement calculated to achieve the same purpose may be substituted for the specific implementations shown. This disclosure is intended to cover any and all adaptations or variations of various implementations. Combinations of the above implementations, and other implementations not specifically described herein, will be apparent to those of skill in the art upon reviewing the above description.

What is claimed is:

1. A system configured to detect a stroke event based on a measurement of one or more physiological parameters from a sole of a foot, the system comprising:
a physiological sensor comprising:
a blood volume sensor;
a shoe insert or a sock configured to integrate a stimulus source and the physiological sensor, wherein the shoe insert or the sock is configured to position the physiological sensor for measurement of the one or more physiological parameters from the sole of the foot of a person; and
one or more hardware processors communicatively coupled to the physiological sensor, wherein the one or more hardware processors are configured to:
output a first activation signal to the stimulus source configured to heat the sole of the foot;
measure one or more physiological parameters from the blood volume sensor responsive to heating the sole of the foot; and
determine a stroke event based at least on the measurement of the one or more physiological parameters.

2. The system of claim 1, wherein the stimulus source comprises a heater trace.

3. The system of claim 2, wherein the heater trace covers a substantial portion of the sole of the foot.

4. The system of claim 2, wherein a heater trace covers a top and bottom portion of the foot.

5. The system of claim 2, wherein the heater trace comprises an opening configured to house a physiological sensor.

6. The system of claim 5, wherein the heater trace covers a substantial portion of the sole of the foot except for the opening.

7. The system of claim 2, wherein the heater trace is positioned in a proximity of an ankle of the person.

8. The system of claim 1, wherein the physiological sensor is a temperature senor.

9. The system of claim 1, further comprising a battery positioned in a proximity of an ankle of the person.

10. The system of claim 1, wherein the physiological sensor is positioned symmetrical along a longitudinal axis of the foot.

11. The system of claim 1, wherein the physiological sensor is symmetrical along a transverse axis of the foot.

12. The system of claim 1, wherein the physiological sensor is intertwined with a fabric of the sock.

13. The system of claim 1, wherein the physiological sensor is sewn to the sock.

14. A system for measuring a stroke event in a person from a site of a foot, the system comprising:
a body portion configured to be positioned on a shoe and to monitor a plurality of skin surface sites of the person;
a stimulus source having a surface area to provide stimulus;
a physiological sensor comprising a blood volume sensor;
a heat source configured to be positioned below a sole of the person; and
at least one processor communicatively coupled to the body portion and configured to:
output a first activation signal to the stimulus source;
output a second activation signal to the blood volume sensor once the at least one of the plurality of skin surface sites reaches a target offset temperature from a baseline temperature;
receive an output signal from the blood volume sensor; and
determine a stroke event based at least on the output signal.

15. The system of claim 14, wherein the body portion is positioned on a top portion of the shoe.

16. The system of claim 14, wherein the heat source comprises heater trace.

17. The system of claim 16, wherein a substantial portion of the sole of the person contacts the heater trace.

18. The system of claim 16, wherein the heater trace is integrated with a shoe insert.
